(19) United States
(12) Patent Application Publication
(10) Pub. No.: US 2010/0179170 A1
(43) Pub. Date: Jul. 15, 2010

(54) HETEROGENEOUSLY CONFIGURED MULTIPARTICULATE GASTROINTESTINAL DRUG DELIVERY SYSTEM

(76) Inventors: Lisa Claire Du Toit, Florida (ZA); Michael Paul Danckwerts, Bedfordview (ZA); Viness Pillay, Sandton (ZA); Shivaan Cooppan, Durban (ZA); Yahya Essop Choonara, Lenasia (ZA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/312,843

(22) PCT Filed: Dec. 3, 2007
(86) PCT No.: PCT/IB2007/003727

§ 371 Date: Mar. 4, 2010

(30) Foreign Application Priority Data
Dec. 5, 2006 (ZA) 2006 08301

(51) Int. Cl. A61K 31/496; A61K 31/44; A61P 31/04

(52) U.S. Cl. 514/254.11; 514/354

(57) ABSTRACT

This invention relates to a heterogeneously configured multiparticulate drug delivery system for gastrointestinal delivery of at least one or a combination of active pharmaceutical compositions. The system comprises a multiplicity of enterosoluble or gastrosoluble multiparticulates loaded with the active pharmaceutical composition or compositions for the site-specific delivery of said active pharmaceutical composition or compositions to a specific region in the gastrointestinal tract of a human or animal body. The system can be supplied as reconstitutable granules which are reconstituted immediately before oral administration.

### FIELD OF THE INVENTION

This invention relates to the design and development of a heterogeneously configured multiparticulate pharmaceutical dosage form, more particularly; to a pharmaceutical dosage form suitable for the delivery of at least one or a combination of active pharmaceutical compositions in the gastrointestinal tract of a human or animal body.

### BACKGROUND TO THE INVENTION

Certain difficulties are experienced when endeavouring to administer, orally, acid-sensitive active pharmaceutical compositions to various regions of the gastrointestinal tract, more particularly the lower gastrointestinal tract, as such active pharmaceutical compositions must first pass through the acidic environment of the upper gastrointestinal tract in the stomach. Similarly, difficulties are experienced when an active pharmaceutical composition that is destined for the lower gastrointestinal tract has characteristics which render it desirable to reduce its release or its retention time in the upper gastrointestinal tract. Such active pharmaceutical compositions are those which affect gastric performance or cause local irritations of the gastric mucosa.

Furthermore, difficulties are also experienced when two or more active pharmaceutical compositions are required to be delivered to either the upper or lower gastrointestinal tract as part of a standard regimen where the said active pharmaceutical compositions may have a deleterious interaction between at least two of the active pharmaceutical compositions that may result in reducing its release, its retention time or bioavailability in the desired region of the gastrointestinal tract. Such active pharmaceutical compositions may also be those which affect gastric performance or cause local irritations of the gastric mucosa.

Oral administration of active pharmaceutical compositions, more particularly over-the-counter or non-prescription pharmaceuticals and active pharmaceutical compositions that are administered over a prolonged period of time is preferred over other methods of administration because it is non-invasive. This does, however, present manufacturers of pharmaceuticals with the above-mentioned difficulties. Attempts to overcome or reduce the above-mentioned difficulties have, in relatively recent times, centred on encapsulation of the active pharmaceutical composition with a polymeric coating which is not dissolved in the upper gastrointestinal tract and, consequently, passes through the upper gastrointestinal tract and into the lower gastrointestinal tract where it is dissolved and the active pharmaceutical composition is released.

While the above-described coated formulations or drug delivery systems can be used quite effectively where the dosage form is a single large or several smaller tablets more often than not this is not the case particularly where the target of the active pharmaceutical composition is a relatively small region of the gastrointestinal tract and where a large tablet may pass by without dissolving completely. To counter this, the active pharmaceutical composition is delivered in the form of a multiplicity of small beads. The beads are formed by coating an inert starch or sugar core with the active pharmaceutical composition which is dissolved in a suitable solvent and sprayed onto the core then coating the active pharmaceutical composition with a sealing polymer which is also sprayed. Up to one thousand of these beads may be administered as a single dose.

While the above-described delivery system is effective it is expensive to produce. Firstly, the size of the core is important for if cores are too large there is less surface area available for applying the active pharmaceutical composition layer and this result in a thicker active pharmaceutical composition layer with consequent manufacturing problems for an intensive drying step is required to reduce residual solvent levels in the active pharmaceutical composition layer. Conversely, while a smaller core has a larger total surface area for coating resulting in a thinner active pharmaceutical composition layer and a far less intensive drying step, cores which are too small tend to agglomerate during the coating process.

Secondly, the actual coating process is expensive for it uses relatively complex equipment and, to facilitate the process air in the equipment must be heated. The spraying process is also repeated, once to form the active pharmaceutical composition layer and the second time to form the seal coating layer.

A final step in the above process is to introduce a predetermined number of beads, based, usually, on the weight of the bioactive, into gelatine or similar capsules which can be swallowed relatively easily. This too adds a step in the manufacturing process which adds to the production time and to the costs of the finished product.

The term “multiparticulate” and “multiparticulates” when used in this specification are intended to be used as a generic term for a heterogeneously configured multiparticulate system, preferably a multiparticulate system that may or may not be enterosoluble or, alternatively, may or may not be gastrosoluble intended for the gastrointestinal delivery of at least one or a combination of active pharmaceutical compositions.

### OBJECT OF THE INVENTION

It is an object of this invention to provide a heterogeneously configured multiparticulate system for gastrointestinal delivery of at least one or a combination of active pharmaceutical compositions which, at least partly, alleviates the above-mentioned difficulties and, to provide a means of crosslinking so as to improve the physicochemical and physicomechanical properties of the multiparticulates to modulate drug release, and to an approach of manufacture and improved drug entrapment efficiency of multiparticulate systems for gastrointestinal pharmaceutical delivery.

### SUMMARY OF THE INVENTION

In accordance with this invention there is provided a heterogeneously configured multiparticulate drug delivery system for gastrointestinal delivery of at least one or a combination of active pharmaceutical compositions, said heterogeneously configured multiparticulate system comprising a multiplicity of enterosoluble or gastrosoluble multiparticulates loaded with said active pharmaceutical composition or compositions for the site-specific delivery of said active pharmaceutical composition or compositions to a specific region in the gastrointestinal tract of a human or animal body.

There is further provided for the active pharmaceutical composition or compositions to be delivered to the small intestine of a human of animal body.

There is also provided for the drug delivery system to incorporate a combination of two or more active pharmaceutical compositions, for the regions of the gastrointestinal tract to which said active pharmaceutical compositions are delivered to be located in the small, alternatively large intestine or stomach or esophagus of the human or animal body and for the multiparticulates to be enterosoluble and/or gastrosoluble depending on the delivery site of the active pharmaceutical composition or compositions.

There is further provided for the multiparticulates to be gastric fluid soluble, alternatively resistant to dissolution in gastric fluid, or alternatively for the multiparticulates to be reconstitutable multiparticulates which disintegrate rapidly in tepid water to form a gel network which, in use, suspends the said active pharmaceutical composition or compositions loaded into the multiparticulates immediately prior to administration, preferably oral administration.

A heterogeneously configured multiparticulate system for the site-specific delivery of one or a combination of active pharmaceutical compositions into the gastrointestinal tract of a human or animal body wherein the multiparticulates are reconstitutable multiparticulates which disintegrate rapidly in tepid water to form a gel network which, in use, suspends the said active pharmaceutical composition or compositions loaded into the multiparticulates.

The invention extends to a pharmaceutical dosage form comprising a heterogeneously configured multiparticulate drug delivery system for gastrointestinal delivery of at least one or a combination of active pharmaceutical compositions, said heterogeneously configured multiparticulate system comprising a multiplicity of enterosoluble or gastrosoluble multiparticulates loaded with said active pharmaceutical composition or compositions for the site-specific delivery of said active pharmaceutical composition or compositions to a specific region in the gastrointestinal tract of a human or animal body.

There is also provided for the pharmaceutical dosage form or multiparticulates to be formed from a polymeric material and for the polymeric material to be a pH-sensitive polymer demonstrating solubility in intestinal fluid above a pH of 4.0, but preferably above a pH of 5.0.

There is further provided for the pH-sensitive polymer to interact and swell minimally in the presence of water at low pH, ionise, swell and dissolve in water at high pH.

There is also provided for the pH-sensitive polymer to be crosslinked in a desirable electrolyte/salt solution with electrolytes/salts chosen but not limited to from among the list of crosslinking agents, preferably from the Hofmeister Series of salts.

There is also provided for the pH-sensitive polymer to be a polymethacrylate-type polymer that is crosslinked to form a series of heterogeneously configured multiparticulates also referred to as multiparticulates.

There is also provided for the pH-sensitive polymer to be carboxylated and contain mixed acid and ester functional groups.

Further according to the invention, suitable pH-sensitive polymers are those that may possess acidic side groups and which demonstrate at least partial solubility in aqueous solutions, such as water, buffered salt solutions, or alkaline solutions. Such acidic acid groups include, but are not limited to, the carboxylic acid moeity, possessing the propensity to interact with suitable cations.

Further, according to the invention, suitable pH-sensitive polymers are enteric polymers possessing carboxylic acid and ester groups on the polymer backbone.

The polymers are selected from the group consisting of but not limited to: methacrylic acid-based polymers, preferably methacrylic acid and ethyl acrylate copolymers (Eudragit® L30D, Eudragit® L100-55) and methacrylic acid and methyl methacrylate copolymers with varying monomer ratios (Eudragit® L100, Eudragit® S100), preferably a poly(methacrylic acid-co-ethylacrylate) copolymer; phthalate-based enteric polymers, preferably cellulose acetate phthalate (Aquateric®) and polyvinyl acetate phthalate (Coateric®); and hydroxypropyl methylcellulose acetate succinate (Aqoat®) and for the copolymer to be a poly(methacrylic acid-co-ethylacrylate) copolymer.

There is also provided for the active pharmaceutical composition to be an acid-sensitive active pharmaceutical composition selected from the group comprising: active pharmaceutical compositions which are unstable or degraded at acidic pH, preferably enzymes, proteins, and macrolide antibiotics such as erythromycin; active pharmaceutical compositions affecting gastric performance; active pharmaceutical compositions causing local irritation of the gastric mucosa, preferably valproic acid and alternatively NSAIDs such as diclofenac and acetylsalicylic acid; active pharmaceutical compositions for which intestinal targeting is required for attainment of adequate concentrations in the lower gastrointestinal tract and bioavailability, preferably 5-aminosalicylic acid, alternatively prodrugs of mesalazine and sulfasalazine; and active pharmaceutical compositions which accelerate the degradation of other active pharmaceutical compositions in the gastrointestinal tract, preferably isoniazid, rifampicin, pyrazinamide, alternatively didanosine and ketoconazole.

The invention extends to a method of forming a multiparticulate system for gastrointestinal delivery of the above-described orally administered multiparticulates comprising inducing separation or salting-out of the pH-sensitive polymer as a polymer-rich enteric film and ionotropically crosslinking the internal multiparticulate matrix following extrusion and curing of a partially neutralized aqueous dispersion of the copolymer into a concentrated electrolyte solution.

There is further provided for the preferred anions for salting-out and inducing separation of the enteric polymer to be pharmaceutically acceptable anions which, in use, demonstrate effectiveness in accordance with the Hofmeister series and, preferably, are selected from the group consisting of: SO₄²⁻, HPO₄²⁻, F⁻, CH₃COO⁻, Cl⁻, Br⁻, and NO₃⁻.

There is also provided for preferred cations for crosslinking the internal multiparticulate matrix with acidic side groups to be divalent or trivalent pharmaceutically acceptable cations which, preferably, are selected from the group consisting of: Ca²⁺, Zn²⁺, Ba²⁺, Mg²⁺, Cu²⁺, and Al³⁺.

There is also provided for the salting-out and crosslinking agent to be a complex salt, preferably zinc sulfate heptahydrate (ZnSO₄7H₂O).

There is also provided for a multiple crosslinking and curing steps with the preferred electrolytes for crosslinking the internal multiparticulate matrix with acidic side groups to be mono- bi- or trivalent pharmaceutically acceptable electrolytes which, preferably, are selected from the group consisting of: Ca²⁺, Zn²⁺, Ba²⁺, Mg²⁺, Cu²⁺, and Al³⁺.

There is further provided for the method to be conducted in a spray-drying apparatus, for the drying chamber to be saturated with the salting-out and crosslinking electrolyte, followed by controlled pumping of the drug-loaded polymeric aqueous dispersion into the drying chamber with droplet formation by rotary atomisation.

There is further provided for the method to be conducted in a spray-drying apparatus, for the drying chamber to be saturated with the salting-out and crosslinking electrolyte, followed by controlled pumping of the drug-loaded polymeric aqueous dispersion into the drying chamber with droplet formation by rotary atomisation.

There is further provided for the method to be conducted in a customized dripper, for the receiving chamber to be saturated with the salting-out and crosslinking electrolyte, followed by controlled pumping of the drug-loaded polymeric aqueous dispersion into the chamber with droplet formation by a customized dripper system.

There is also provided for the electrolyte-saturated air-filled chamber to be maintained at the designated temperature setting for optimum annealing of the plasticized multiparticulate film and matrix.

According to a further aspect of the invention, the multiparticulates will be delivered as dispersible multiparticulates, which are reconstitutable from a dry suspension system incorporating at least one more active pharmaceutical composition intended, in use, for instant-release.

According to a further aspect of the invention, the multiparticulates will also comprise two separate heterogeneously configured multiparticulate systems delivered as a single pharmaceutical dosage form to a human or animal body and, preferably, for each set of multiparticulates to have different mechanisms of active pharmaceutical composition release behavior for delivering, in use, at least one desirable active pharmaceutical composition or a set of active pharmaceutical compositions that may form part of a standard treatment regimen and may also be known to have a deleterious interaction between the said active pharmaceutical compositions when delivered simultaneously to a human or animal body.

There is also provided for the dry heterogeneously configured multiparticulates, preferably a suspension, to be provided as reconstitutable multiparticulates, preferably granules, for extemporaneous dispensing which may be freshly reconstituted prior to administration to a patient by adding a suitable solubilizing agent/solvent, preferably water.

There is further provided for the reconstitutable multiparticulates to be prepared by mixing an orally administrable hydrolytically stable active pharmaceutical composition intended for instant-release in the gastrointestinal tract, and the suspension and granulation adjuvants, preferably according to a wet granulation technique.

There is also provided for the oral pharmaceutical granules for reconstitution as a suspension to contain at least one hydrophilic gel-forming viscosity-enhancing agent for adequate suspension of the active pharmaceutical composition loaded into multiparticulates and the incorporated active pharmaceutical composition.

There is further provided for the gel-forming agent or agents to be a pharmaceutically acceptable viscosity agent, preferably selected from the group consisting of: xanthan gum, hydroxypropylmethyl cellulose, methylcellulose, carageenan, carboxymethyl cellulose, microcrystalline cellulose, polyvinylpyrrolidone, soluble starches and carbomers.

There is also provided for the gel-forming agent or agents to disperse and gel rapidly to form a suspension possessing the necessary properties for extemporaneous use on reconstitution in tepid water.

Preferably the suspension system is a hydrophilic polymer composite system comprising two suspending and gel-forming agents, which includes, but is not limited to, the combination of a polysaccharide gums such as xanthan, guar gum, or carrageenan and a soluble starch-based system. Preferably, the soluble starch demonstrates dual functionality as a hydrophilic suspending agent and granule disintegrant. Preferably, the soluble starch is a pregelatinised starch or sodium starch glycolate.

There is further provided for additional adjuvants to be included in the reconstitutable granules and for said agents to include such agents as are required for an adequate extemporaneous formulation, which include a water-soluble lubricant, granulating agent and sweetening agent, which may include any water-soluble pharmaceutically acceptable agent demonstrating acceptable performance in the aforementioned functions.

### BRIEF DESCRIPTION OF ONE EMBODIMENT OF THE INVENTION

The above and additional features of the invention will become evident from the below-described non-limiting example which describes a delivery system for facilitating gastrointestinal delivery of rifampicin and isoniazid upon co-administration as a fixed-dose combination. Other such examples included ketoconazole and didanosine. The following figures are referred to in the example:

FIG. 1: Schematic of proposed (a) inter- and (b) intra-molecular ionic interactions (‘salt-bridges’) between the anionic poly(methacrylic acid-co-ethylacrylate) copolymer (MAEA) and cationic agent;

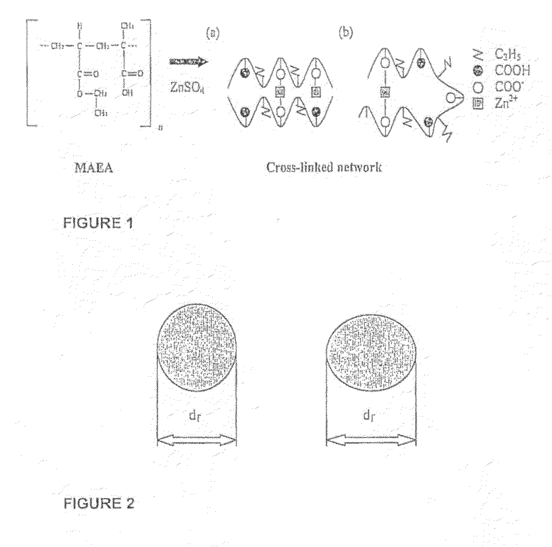

FIG. 2: Particle orientation for determination of shortest and longest Feret's diameters (d_{f});

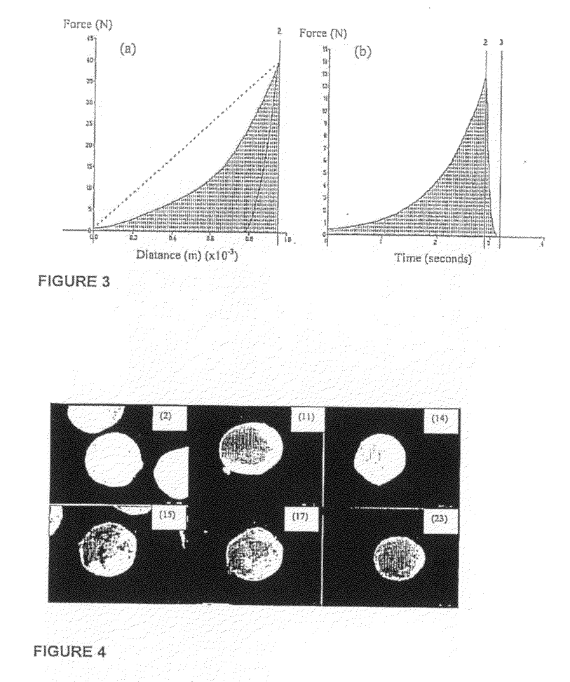

FIG. 3: Typical textural profiles for the measurement of (a) deformation energy (upward gradient) and matrix hardness (AUC) and (b) resilience;

FIG. 4: Stereomicrographs (16× magnification) of multiparticulate formulations 2, 11, 14, 15, 17, 23;

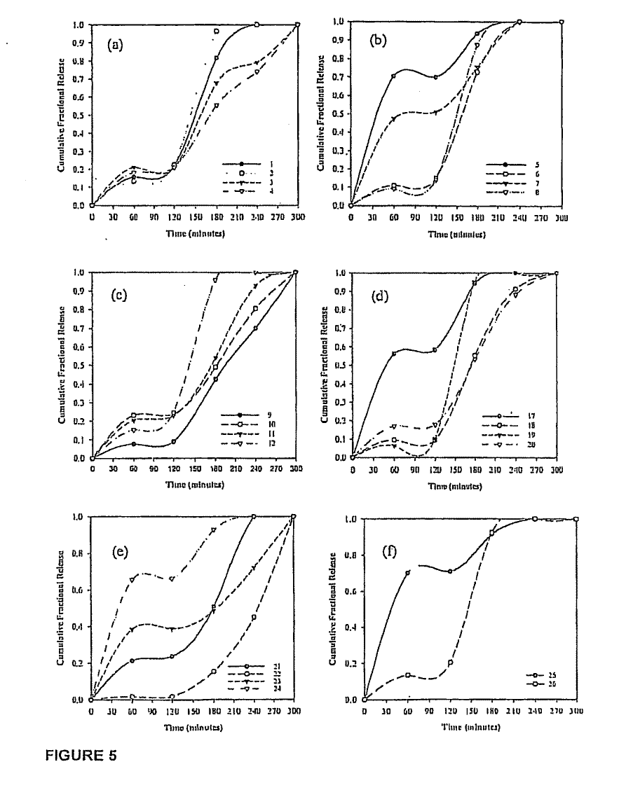

FIG. 5: Composite release profiles (a-f) of the multiparticulate formulations in acidic (pH 1.2) and phosphate buffered media (pH 6.8) (S.D.<±0.040 in all cases;

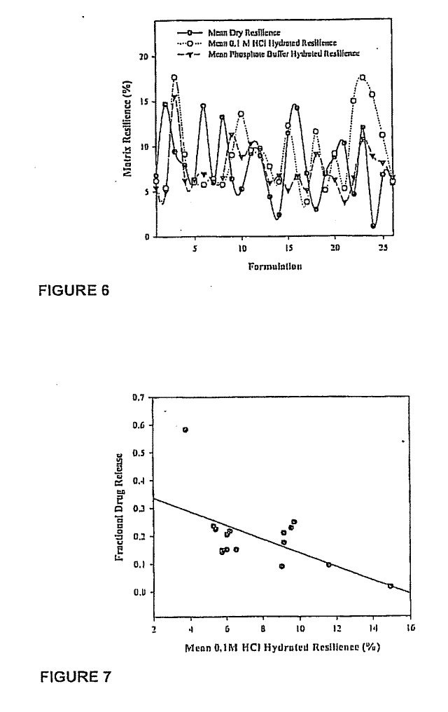

FIG. 6: Variable resilience of multiparticulate formulations in the dry and hydrated state;

FIG. 7: Relationship between fractional drug release and acid-hydrated resilience;

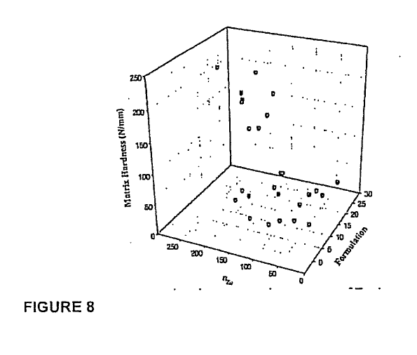

FIG. 8: 3-D scatter plot of matrix hardness vs. molar amount of Zn (n_{Zn}) vs. formulation;

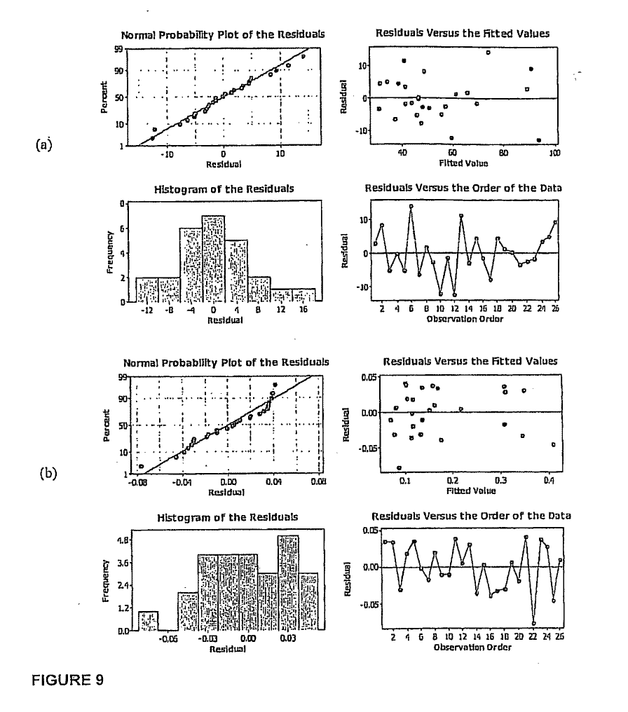

FIG. 9: Residual Plots for (a) DEE and (b) MDT;

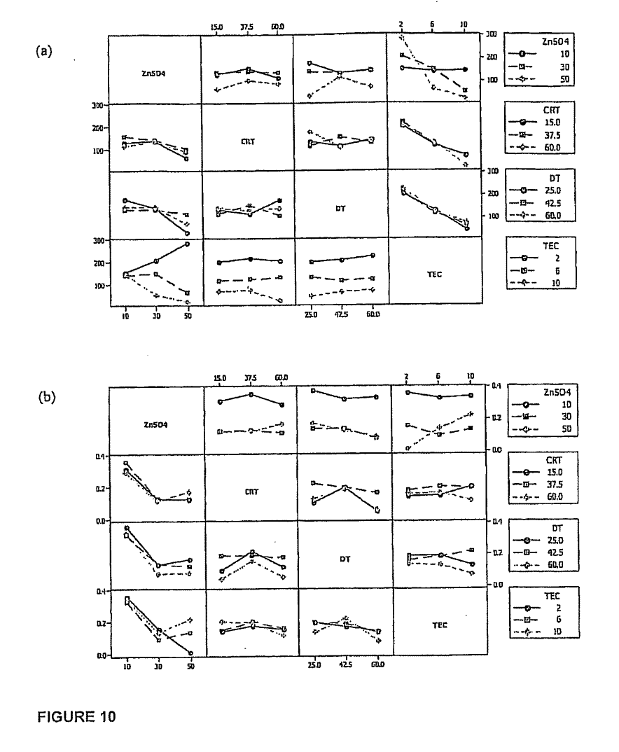

FIG. 10: Interaction plots for (a) n_{Zn}, and (b) MDT;

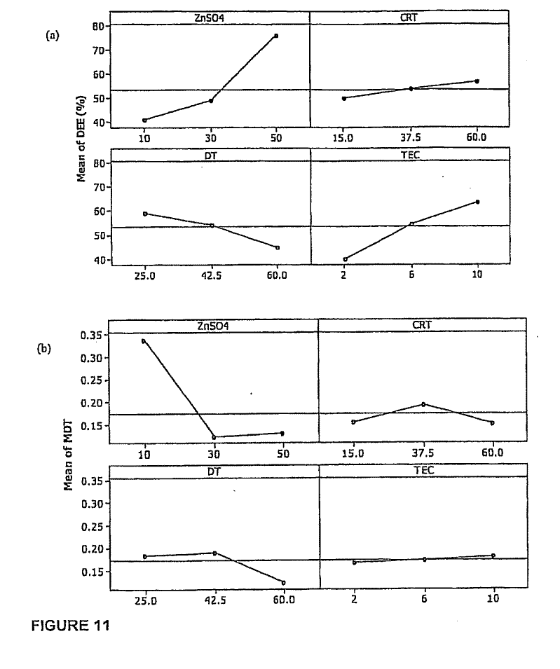

FIG. 11: Main effects plots for (a) DEE and (b) MDT;

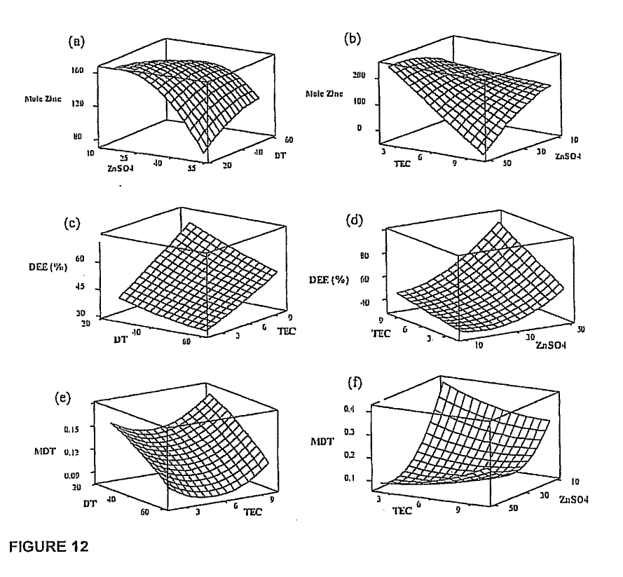

FIG. 12: Response surface plots for n_{Zn}, DEE and MDT;

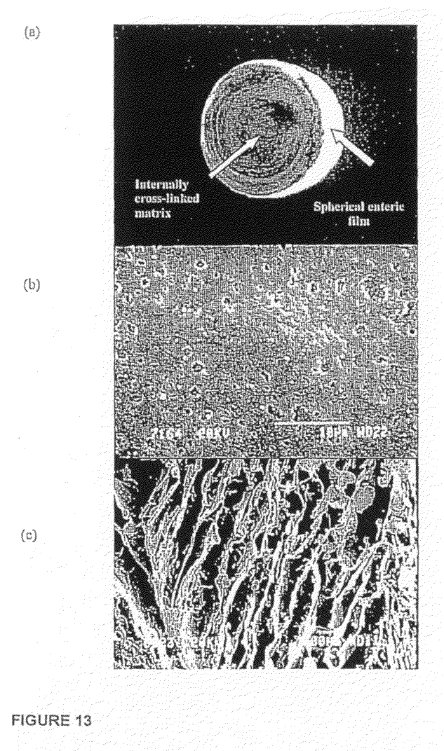

FIG. 13: Stereomicrographs and corresponding scanning electron micrographs of multiparticulate formulation 22 depicting (a) cross-section of multiparticulates (b) the patent spherical enteric film at 3000× magnification and (c) the crosslinked internal matrix at 100× magnification;

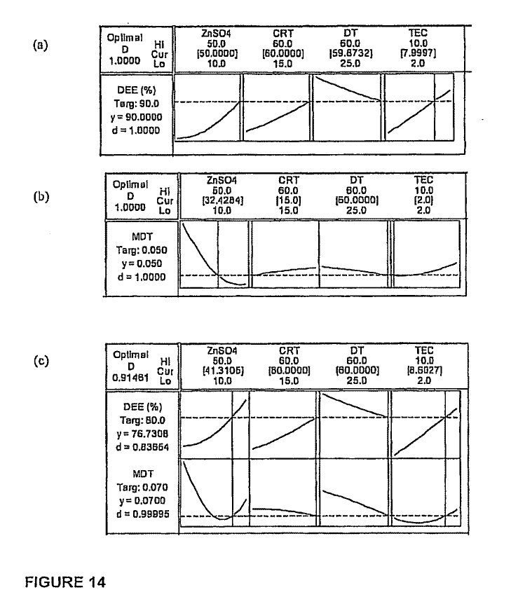

FIG. 14: Optimization plots delineating factor settings and desirability values for optimal formulations (a) F1; (b) F2; and (c) F3;

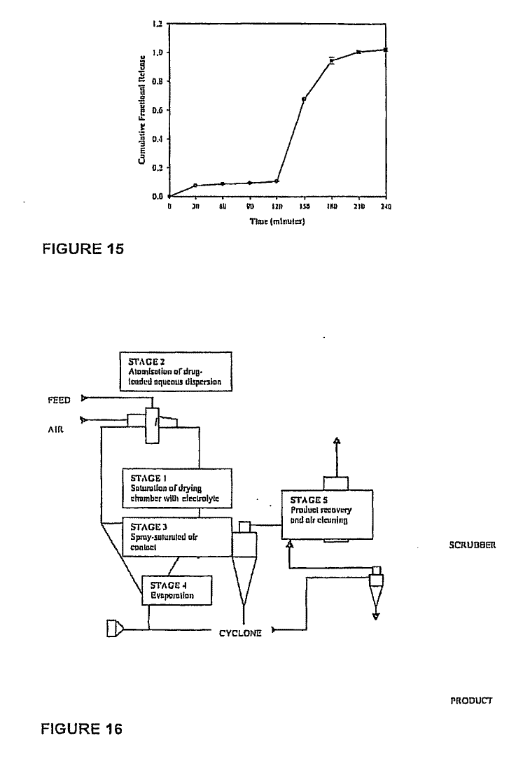

FIG. 15: Composite release profile of isoniazid from optimum formulation (F3); and

FIG. 16: Spray-dryer configuration and process staging.

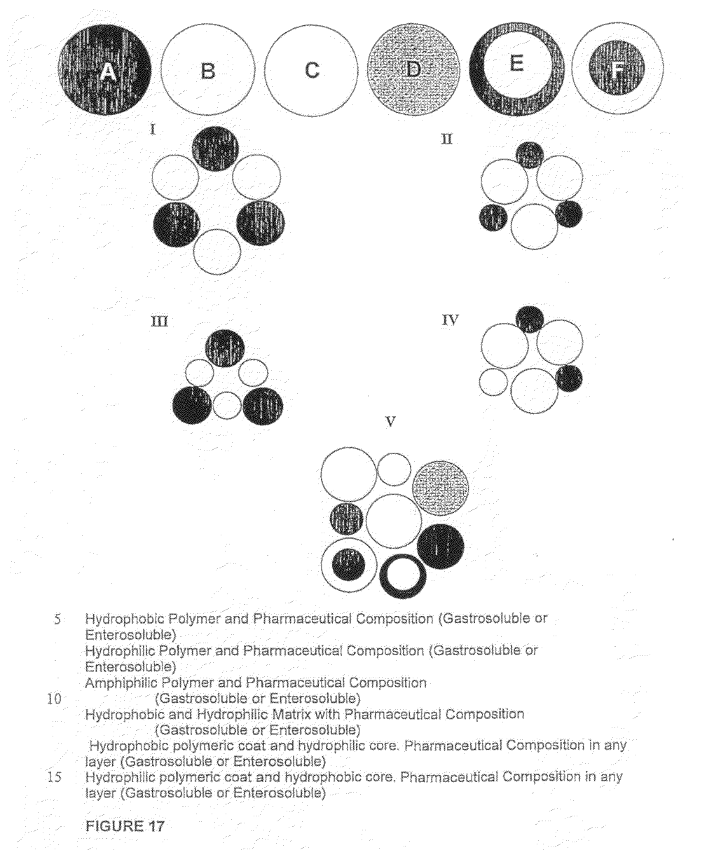

FIG. 17: Schematics displaying various heterogeneously configured multiparticulate gastrointestinal drug delivery systems.

### DETAILED DESCRIPTION OF ONE EMBODIMENT OF THE INVENTION

In this example ionotropically crosslinked multiparticulates for delivery of isoniazid (INH) to the small intestine were developed via a response surface methodology (RSM) for the design and optimization of the formulation and processing variables. This was to facilitate differentiated gastrointestinal delivery of rifampicin (RIF) and INH upon co-administration as a fixed-dose combination. A four-factor, three-level (3⁴) Box-Behnken statistical design was constructed. The concentration (% w/v) of zinc sulfate (ZnSO₄) salting-out and cross-linking electrolyte, the cross-linking reaction time (CRT), the drying temperature and the concentration (% w/w) of triethyl citrate (TEC) plasticizer were varied for determination of the effect of the experimental factors on the molar amount of zinc (n_{Zn}) incorporated in the crosslinked multiparticulates, drug entrapment efficiency (DEE), and mean dissolution time (MDT) at t₂ₕ in acidic media (0.1 M HCl). Complexometric determination of zinc cations (Zn²⁺) revealed that 23.70 to 287.89 moles of Zn²⁺ per mole of methacrylic acid copolymer was implicated in cross-link formation. Entrapment efficiencies of 27.92% to 99.77% were obtained. The ability of the multiparticulates to slow drug release in acidic media varied greatly—drug release at t₂ₕ ranged from 1.67% to 73.04%. Polymeric disintegration and drug release in alkaline media was comparatively rapid for all variants owing to hydration of the carboxylic acid groups of the methacrylic acid copolymer and removal of Zn²⁺ from the matrix due to sequestration by phosphate ions. The salting-out and cross-linking agent significantly affected n_{Zn} (p=0.034) and the DEE (P=0.000), as did the concentration of plasticizer employed (p=0.000 and p=0.002 respectively). High drying temperatures (>42.5° C.) also significantly improved DEE (p=0.029). ZnSO₄ had a significant effect on the MDT (p=0.000). A significant interaction effect was observed between ZnSO₄ and TEC on n_{Zn} (p=0.005) and on the MDT (p=0.035). Additional experiments performed at the optimal variable settings confirmed the validity and reliability of the proposed models in predicting the drug entrapment and dissolution behaviour of the multiparticulates. Industrial scale-up of the described process was configurationally staged. Delivery of the optimum INH-loaded multiparticulate system as a dispersible multiparticulate system in combination with RIF was also delineated.

The formation and properties of polymeric muitiparticulates ionotropically crosslinked via multivalent ions for modified drug delivery will depend on the concentrations and distribution of the ions incorporated within the polymer, which in turn is affected by the duration of exposure of the polymer to the salting-out and cross-linking solution. The polymeric chains are crosslinked via cations by the formation of complexes liganded with more than one polymer group creating intramolecular and/or intermolecular cross-links.¹ The inclusion of a plasticizer will also have a distinctive effect on the characteristics of the polymeric matrices due to its influence on the polymer's melt viscosity, glass-transition temperature (T_{g}), minimum film-forming temperature (MFT) and elastic modulus as a result of the plasticizer's ability to weaken polymeric intermolecular attractions and to increase the polymers free volume.^{2,3}

Statistical experimental designs are strongly recommended in identifying critical formulation variables in the development of modified-release drug delivery systems.⁴ In the implementation of a novel salting-out and cross-linking method for the formulation and design of ionotropically crosslinked multiparticulates, for delivery of a water-soluble drug to the small intestine, the use of a response surface methodology (RSM) allows for the generation of mathematical models to adequately describe or predict the drug entrapment and release behaviour of the multiparticulates.

The fabrication of crosslinked multiparticulates in a single processing step (precluding the use of expensive machinery and organic solvents) is an alternative approach to the standard technique for manufacturing modified-release multiparticulates, which consists of coating drug-containing granules or beads with aqueous colloidal latex or pseudolatex polymeric dispersions. However, a problem associated with enteric-coated formulations made of aqueous disperse systems or solutions is the lack of resistance against gastric fluid and the reportedly more rapid diffusion of water-soluble drug through films prepared from aqueous solutions than through organic-solvent-based films.⁵ Bianchini et al.⁶ demonstrated the poor performance of enteric-coated dosage forms containing a water-soluble substance; these did not pass the USP 24 test unless insulation of the cores was undertaken by sub-coating barriers or by employing twice the amount of coating. It is contemplated that fabrication of an optimal crosslinked enteric-polymer matrix system incorporating a water-soluble drug would achieve improved gastro-resistance of the multiparticulate system.

The polymeric material used in the present study to achieve enteric properties was poly (methacrylic acid-co-ethylacrylate) copolymer, which is soluble in intestinal fluid above pH 5.5 due to ionization of its carboxylic acid groups. However, alternative carboxylated pH-sensitive polymers containing mixed acid and ester functional groups, demonstrating solubility by ionisation and swelling in intestinal fluid above a pH of at least 4.0, but interacting and swelling minimally in the presence of water at low pH, have been employed for multiparticulate fabrication as described in this study. The carboxylic acid moeity, in particular, possesses the propensity to interact with suitable cations. A further pre-requisite for adequate cross-linking under these conditions was demonstration of at least partial solubility in aqueous solutions, such as water, buffered salt solutions, or alkaline solutions.

pH-sensitive polymers investigated preliminarily for the aforementioned purpose encompassed common enteric polymers including the methacrylic acid-based polymers such as methacrylic acid and ethyl acrylate copolymers (Eudragit®L 30D, Eudragit®L 100-55), methacrylic acid and methyl methacrylate copolymers with varying monomer ratios (Eudragit®L 100, Eudragit®S100), the phthalate-based enteric polymers such as cellulose acetate phthalate (Aquateric®) and polyvinyl acetate phthalate (Coateric®), in addition to other enteric polymers such as hydroxypropyl methylcellulose acetate succinate (Aqoat®). For the purposes of this investigation, poly(methacrylic acid-co-ethylacrylate) copolymer was selected for the identification of an optimum system, demonstrating reproducible performance.

The salted-out and crosslinked multiparticulate matrices were formed by inducing separation of the anionic polyelectrolyte as a polymer-rich enteric film (the ‘salting-out’ phenomenon) and ionotropically cross-linking the internal multiparticulate matrix (FIG. 1) following extrusion and curing of an aqueous dispersion of the polymer into a concentrated electrolyte solution. Electrolytes comprising various cations and anions were investigated preliminarily, with the preferred anions for salting-out and inducing separation of the enteric polymer being pharmaceutically acceptable anions which include SO₄²⁻, HPO₄²⁻, F⁻, CH₃COO⁻, Cl⁻, Br⁻, and NO₃⁻, demonstrating effectiveness in accordance with the Hofmeister series. The preferred cations for crosslinking the internal multiparticulate matrix with acidic side groups are divalent or trivalent pharmaceutically acceptable cations, which include, Ca²⁺, Zn²⁺, Ba²⁺, Mg²⁺, Cu²⁺, and Al³⁺.

Zinc sulfate heptahydrate (ZnSO₄.7H₂O) was selected as the salting-out and cross-linking agent, demonstrating superior performance in comparison to other salts evaluated in preliminary investigations owing to the favourable salting-out capabilities of the sulfate anion (SO₄²⁻) in accordance with the Hofmeister series and the superior cross-linking capabilities of the Zn²⁺ for the methacrylic acid copolymer. In addition, the salt demonstrates high water solubility (1 in 0.6 water).⁷

The methacrylic acid ethyl acrylate copolymer is a synthetic polymer demonstrating excellent biocompatibility, and is suitable for ionotropic cross-linking in this manner to form interconnected matrices (FIG. 1). As anionic polyelectrolytes, they have charged carboxylic acid side groups and although they are practically insoluble in water, they are soluble in solutions of 1 M NaOH upon neutralization of carboxyl groups.⁸ The water-dispersed polymer with charged side groups was crosslinked by reaction with a solution of cations such as Zn²⁺.

An approach to multi-step crosslinking of the polymethacrylates for superior physicochemical and physicomechanical stability and further modulation of drug release was also explored.

Enteric-release is prescribed for the delivery of acid-sensitive bioactives belonging to the following categories: bioactives unstable or degraded at acidic pH (e.g. enzymes, proteins, macrolide antibiotics such as erythromycin) bioactives affecting gastric performance, bioactives causing local irritation of the gastric mucosa (e.g. valproic acid, NSAIDs such as diclofenac and acetylsalicylic acid), bioactives for which intestinal targeting is required for attainment of adequate concentrations in the lower gastrointestinal tract (e.g. 5-aminosalicylic acid, prodrugs of mesalazine and sulfasalazine), bioactives which accelerate the degradation of other bioactives in acidic media (e.g. INH, pyrazinamide and didanosine and ketoconazole).

The model drug incorporated within the enteric-release system was INH, the most active drug for the treatment of tuberculosis (TB) caused by susceptible strains, which is administered in combination with RIF during the intensive and continuation phases of anti-TB chemotherapy.⁹ The rationale for targeted delivery of this drug to the small intestine arises from the urgent need to segregate the delivery of RIF and INH upon co-administration, such that INH is not released in the stomach owing to the induction of accelerated hydrolysis of RIF in acidic medium to the poorly absorbed insoluble 3-formyl rifamycin SV in the presence of INH.^{10,11,12,13}

In the present study, a Box-Behnken design was employed for the generation of quadratic response surfaces and construction of second order polynomial models for the prediction of the multiparticulate behavior in terms of the independent variables investigated. This will facilitate a mechanistic evaluation of possible correlations between pertinent processing factors such as the concentration of ZnSO₄, CRT, TEC level and DT employed in the formulation of the multiparticulates on their ability to entrap the drug and target its release to the small intestine for the formulation of an optimal system.^{14.15} The multiparticulate formulations were characterized in terms of their aspect ratio (a shape factor), molar amount of zinc (n_{Zn)} incorporated within the crosslinked matrix, drug loading and drug entrapment efficiency (DEE), fractional isoniazid release and mean dissolution time (MDT) in acidic media at t₂ₕ and textural parameters for each of the polymeric variants (matrix resilience, deformation energy and matrix hardness). Response optimization was then employed to identify an ideal polymeric enteric-release multiparticulate matrix system with the desired drug entrapment and dissolution properties.

Materials used were a Methacrylic Acid-Ethyl Acrylate Copolymer with a monomer molar ratio of 1:1 (EUDRAGIT® L100-55, Methacrylic Acid Copolymer Type C) containing sodium lauryl sulphate (0.7% w/w) and polysorbate 80 (2.3% w/w) as emulsifiers, which was a gift from Röhm Pharma Polymers (Röhm GmbH, Darmstadt, Germany). INH (isonicotinic acid hydrazide, 99% TLC) and TEC 99% was purchased from Aldrich® (Sigma-Aldrich Inc., St. Louis, USA). Sodium hydroxide (NaOH, Mw=40.00 g/mol), zinc sulphate (ZnSO₄. 7H₂O, Mw=287.54 g/mol) and ethylenediaminetetraacetic acid as the sodium salt (EDTA, C₁₀H₁₆N₂O₈, (HOOCCH₂)₂N—CH₂CH₂—N(CH₂COOH)₂, Mw=292.24 g/mol) were obtained from Saarchem (Wadeville, Gauteng, South Africa). All other reagents were of analytical grade and were used as received.

To formulate the multiparticulates the methacrylic acid-ethyl acrylate copolymer was re-dispersed effected by addition of 1M NaOH in order to achieve neutralization of approximately 6 mole-% of the carboxyl groups contained in the polymer. TEC, at various percentage levels, was included as a plasticizer. Dissolution of the water-soluble isoniazid in the aqueous dispersion was achieved under agitation at 500 rpm for 10 minutes with a Heidolph® propeller stirrer (Labotec, Gauteng, South Africa) to obtain a methacrylic acid copolymer:isoniazid ratio of 5:1. The dispersion was vortexed (Vortex Genie-2, Scientific Industries Inc., USA) before further processing to allow for homogenization and the dissipation of any foam induced during re-dispersal. 10 ml of the dispersion was then extruded drop-wise at a rate of 2.0 ml/min, using a flat-tip needle (Terumo®, GmbH; Germany) of 0.80-mm internal diameter, into 100 ml of a gently agitated ZnSO₄ solution, which induced immediate salting-out with formation of spherical enteric coating.

The formed multiparticulates were cured in a dark area for the experimentally determined protracted time intervals to induce cross-linking of the internal matrix. The multiparticulates were then washed twice with double-deionized water (100 mL) to remove any unincorporated electrolyte and then oven-dried at different temperature settings for 3 hours followed by cooling slowly under ambient conditions (21° C.). Heating of the multiparticulates at elevated temperatures below the crystalline melting point is known to result in subsequent annealing, which may cause a significant increase in the crystallinity of the enteric polymer, as well as relieving stresses.

A method for the formulation of enterosoluble multiparticulates instituting multi-step crosslinking of the polymethacrylates was also explored. Briefly, a 50 mL INH-loaded latex containing 30 mL double deionised water, 20% w/v methacrylic acid-ethyl acrylate copolymer and 5% w/v 1M NaOH was prepared. Triethyl citrate was added as a plasticizer and the entire latex was placed under a Heidolph® propeller stirrer (Labotec, Gauteng, South Africa) for 30 min. INH (6% w/v) was then added with further agitation. Three separate electrolyte solutions were prepared and included two, 25% w/v ZnSO₄ solutions and a combination solution of ZnSO₄ and MgSO₄ in a 1:1 ratio. The latex (10 mL) was added to each of the electrolyte solutions using a novel dripper system. The ZnSO₄ and the combined ZnSO₄+MgSO₄ multiparticulates were left to cure for 15 min. A set of multiparticulates were then removed from the ZnSO₄ solution and immersed in a 25% w/v MgSO₄ solution to cure. Multiparticulates were then washed with double deionised water (100 mL) to remove any unincorporated electrolyte and dried overnight under ambient conditions (21° C.).

A second approach to multi-step crosslinking of the polymethacrylates for superior physicochemical and physicomechanical stability and further modulation of drug release was explored with the addition of a hydrophobic and hydrophilic polymer such as ethylcellulose (EC) and hydroxypropylmethylcellulose (HPMC). Briefly, a 50 mL solution of an INH-loaded latex containing 30 mL double deionised water, 20% w/v Eudragit L100-55 and 5% w/v 1M NaOH. Eudragit powder was weighed and added in specific portions to 30 mL double deionized water. 1M NaOH was then added to the latex in a drop-wise manner to allow for neutralization. Ethylcellulose (20% w/v in methanol) was then incorporated into the latex solution. Triethyl citrate was added as a plasticizer and the entire latex was placed under a Heidolph® propeller stirrer (Labotec, Gauteng, South Africa) for 30 min. INH (6% w/v) was then added with further agitation. Two separate electrolyte solutions were prepared and included one, 25% w/v AlCl₃ solutions and a 25% w/v BaCl₂ solution. The latex (10 mL) was then added to each of the electrolyte solutions in a drop-wise manner. Each curing solution comprised drug-saturated electrolyte solutions (6% w/v) and multiparticulates were left to cure for 10 min. Multiparticulates were then washed thrice with deionised water (500 mL) to remove any unincorporated electrolyte and dried overnight under ambient conditions (21° C.).

It has been demonstrated that multi-step crosslinking has the potential to further modulate drug release. The approach of multiple crosslinking with two or more electrolyte solutions allows for superior crosslinking of the methacrylate polymer with enhanced physicochemical and physicomechanical properties that are able to impart desirable controlled drug release kinetics. The type of electrolyte selected was significant in determining the degree of crosslinking whereby ions with a higher valency provided superior crosslinking. Thus, various formulations combining different permutations of AlCl₃ were investigated as follows: AlCl₃ and CaCl₂, AlCl₃ and BaCl₂, AlCl₃ and ZnCl₂, AlCl₃ and MgCl₂, AlCl₃ and NaCl, AlCl₃ and KCl.

When considering multi-step crosslinking, curing times were found to be crucial. The latex containing Eudragit EL100-55 was added drop-wise into the first electrolyte solution comprising the trivalent salt AlCl₃ and left to cure under darkness for 10 min.

This formed the primary crosslinking base. Thereafter the multiparticulates were removed form the AlCl₃ solution and washed with double deionised water and then added to the second bivalent electrolyte solution and left to cure for the same duration under the same conditions. Following curing, the multiparticulates were then washed in double deionised water and then left overnight to dry under ambient conditions.

Each electrolyte solution comprised the same concentration and cured for the same duration (10-40 min) to ensure an even degree of crosslinking. The intermittent washing of the multiparticulates between electrolyte solutions ensured that no cross contagion of excess electrolyte from one electrolyte solution to the other would occur.

In order to increase the drug entrapment efficiency of the afore-described multi-step methodology, three aspects were explored. Firstly, it was revealed that an increased curing time in a drug-saturated AlCl₃ electrolyte solution increased the potential for drug to be entrapped, consequential of a more efficient crosslinking capacity. Secondly, the addition of the hydrophilic polymer hydroxypropylmethyl cellulose (HPMC) to the latex allowed for more controlled drug release and further aided drug entrapment. Lastly, the addition of the hydrophobic polymer ethylcellulose to the latex was explored. Ethylcellulose was firstly dissolved in methanol/ethanol, and then added to the latex. It was postulated that the addition of ethylcellulose would increase the structural stability of the multiparticulates and aid in the retention of more drug and further modulate drug release. The latex was agitated with a magnetic stirrer that allowed the methanol/ethanol mixture to evaporate. Curing in this instance included two electrolyte solutions, AlCl₃ (first curing solution) and CaCl₂ (second curing solution) with the same curing time and concentrations. The formulations fabricated instituting the multi-step methodology was evaluated for their DEE and drug release behavior.

Optimization of the multiparticulates formulated via inducing the separation and cross-linking of the methacrylic acid copolymer in ZnSO₄.7H₂O was conducted by constructing and analyzing a four-factor, three-level (3⁴) Box-Behnken statistical design on MINITAB®, (V14, Minitab, USA). ZnSO₄ (10-50% w/v), CRT (15-60 minutes), DT (25-60° C.) and TEC (2-10% w/w) were varied (Table 1) for determination of the effect of the experimental factors on n_{Zn}, DEE and MDT in acidic media (0.1 M HCl).

TABLE 1
Factors and levels of independent variables generated by the 3⁴
Box-Behnken Design

| Experimental | ZnSO₄ | | | Plasticizer |
|---|---|---|---|---|
| Formulation | (% ^{w}/ᵥ) | CRT (minutes) | DT (° C.) | (TEC) (% ^{w}/_{w}) |
| 1 | 50 | 60.0 | 42.5 | 6 |
| 2 | 30 | 15.0 | 42.5 | 10 |
| 3 | 30 | 15.0 | 25.0 | 6 |
| 4 | 30 | 37.5 | 42.5 | 6 |
| 5 | 10 | 37.5 | 42.5 | 10 |
| 6 | 30 | 60.0 | 42.5 | 10 |
| 7 | 10 | 60.0 | 42.5 | 6 |
| 8 | 50 | 15.0 | 42.5 | 6 |
| 9 | 30 | 60.0 | 60.0 | 6 |
| 10 | 30 | 60.0 | 25.0 | 6 |
| 11 | 30 | 15.0 | 42.5 | 2 |
| 12 | 50 | 37.5 | 42.5 | 10 |
| 13 | 10 | 37.5 | 25.0 | 6 |
| 14 | 30 | 37.5 | 60.0 | 10 |
| 15 | 30 | 60.0 | 42.5 | 2 |
| 16 | 30 | 37.5 | 25.0 | 10 |
| 17 | 10 | 15.0 | 42.5 | 6 |
| 18 | 30 | 15.0 | 60.0 | 6 |
| 19 | 50 | 37.5 | 60.0 | 6 |
| 20 | 30 | 37.5 | 42.5 | 6 |
| 21 | 30 | 37.5 | 60.0 | 2 |
| 22 | 50 | 37.5 | 42.5 | 2 |
| 23 | 30 | 37.5 | 25.0 | 2 |
| 24 | 10 | 37.5 | 60.0 | 6 |
| 25 | 10 | 37.5 | 42.5 | 2 |
| 26 | 50 | 37.5 | 25.0 | 6 |

The Feret's diameters (d_{f}) and shape of the multiparticulates were investigated by microscopic image analysis using a stereomicroscope (Olympus SZX7, Japan) connected to a digital camera (CC 12) and image analysis system (AnalySIS® Soft Imaging System, GmbH, Germany). Feret's diameter is determined from the mean distance between two parallel tangents to the projected particle perimeter (FIG. 2).
Fifty multiparticulates from each of the formulations were viewed under darkfield at 16× magnification. From the longest and shortest Feret's diameters for each formulation, a shape factor (the aspect ratio) was calculated as follows:

$$Aspect ratio = \frac{{d}_{max}}{{d}_{min}} \qquad [ 1 ]$$

The determination of molar amount of zinc incorporated within the crosslinked matrix, the n_{Zn} was determined by complexometric/chelometric titration of Zn²⁺ with EDTA (ethylenediaminetetraacteic acid, C₁₀H₁₆N₂O₈). EDTA forms very strong 1:1 complexes with divalent and trivalent metal ions depending on solution conditions. The EDTA reacts with the Zn²⁺, to form a chelate as follows:

EDTA+Zn²⁺=ZnEDTA [2]

For the analysis of the amount of Zn²⁺ incorporated within the crosslinked matrix, 0.95 g multiparticulates were hydrated in 25 mL deionized water. Immediately prior to titration, 15 mL of deionized water, 9-10 mL of ammonia/ammonium chloride buffer (pH 10), and 3 drops of Eriochrome Black T were added. The samples were titrated with a standardized solution of 0.01 M EDTA until the pink solution turned light blue.

For determination of the drug loading of the multiparticulates, 100 mg of INH-loaded multiparticulates was placed in a 200 mL conical flask containing 100 mL of 0.2M phosphate buffered saline (PBS), pH 6.8. The multiparticulates were magnetically stirred for 5 hours to promote and ensure erosion and disentanglement of the crosslinked structure to afford liberation and subsequent dissolution of INH. These solutions were filtered through a 0.45 μm membrane filter (Millipore®, Billerica, Md., USA). The filtrates were then made up to 200 mL volumes with the PBS pH 6.8. Aliquots of these solutions were subjected in triplicate to ultraviolet spectroscopy (diode array UV spectrophotometer, Specord 40, Analytik Jena AG, Jena) at 263 nm for analysis (WinASPECT® Spectroanalytical Software, Analytik Jena AG, Jena) following comparison with the standard calibration curves generated for INH in PBS media. Note that it is established at the outset that the polymer solution and/or latex and all other excipients employed in the encapsulation process did not interfere with drug analysis at the reported wavelength. The entrapment capacity was determined by the following empirical relationship:

$$D E E ( \% ) = \frac{\begin{matrix}Actual quantity of drug \\ present in enterospheres\end{matrix}}{\begin{matrix}\begin{matrix}Theoretical quantity of drug loaded \\ into enterospheres\end{matrix} \\ ( actual initial loading dose )\end{matrix}} \times 100 \qquad [ 3 ]$$

Characterization of INH release from the multiparticulates was assessed using a method based on general drug release standard for delayed release (enteric-coated) articles employing the USP 24 apparatus II (paddle apparatus).¹⁶ The six-station dissolution apparatus (Caleva®, Model 7ST) was modified with insertion of a ring-mesh assembly in the dissolution vessel to prevent undue sticking of the particles to the paddle.¹⁷ Each vessel was filled with 500 mL of 0.1 M HCl (pH 1.2) as the initial dissolution media. After 2 hours, the acidic medium was drained from the vessels and replaced with 500 ml PBS (pH 6.8) and samples were withdrawn for a further 3 hours at which time all the formulations had completely dissolved. The collected and filtered samples were diluted and the absorbance measured spectrophotometrically at 265 nm and 263 nm in acidic and phosphate-buffered media respectively for comparison with the standard calibration curves. All tests were performed in triplicate.

A model independent approach was used to compare the dissolution data of the different experimentally synthesized multiparticulates. For this purpose a mean dissolution time (MDT) was calculated for each formulation using the following equation¹⁷.

$$M D T = {\sum }_{t = i}^{n} ti \frac{{M}_{t}}{M ∞} \qquad [ 4 ]$$

Where Mₜ is the fraction of dose released in time ti=(tᵢtₗ₋₁)/2 and M∞ corresponds to the loading dose.

Textural profiling of the multiparticulate formulations was conducted for elucidation of their resilient properties, matrix deformation energy and matrix hardness. A calibrated Texture Analyser (TA.XTplus Texture Analyser, Stable Microsystems®, Surrey, UK) fitted with a 50 kg load cell was employed for the determination of the matrix hardness and deformation energy of unhydrated spheres (using a 2 mm flat-tipped steel probe) and matrix resilience of unhydrated and acid- and base-hydrated multiparticulates (using a 36 mm cylindrical steel probe). The fully integrated data acquisition, analysis and display software (Texture Exponent, Version 3.2) was employed to acquire data at 200 points/second. Studies were conducted at ambient conditions (21±0.5° C.). Results were expressed as the mean of at least three measurements.

The matrix hardness (N/mm), calculated as the gradient of the force-displacement profile during the compression phase (FIG. 3(a)) and deformation energy (N.m or J), calculated as the area under the force-displacement curve (AUC) (FIG. 3(a)); was determined for the unhydrated multiparticulate formulations as per the Texture Analyser settings outlined in Table 2.

TABLE 2
Textural parameters for determination of matrix hardness, deformation
energy and matrix resilience

| | Matrix Hardness | |
|---|---|---|
| | and Deformation | Matrix Resilience |
| Parameter | Energy Settings | Settings |
| Pre-test speed | 1.00 mm/s | 1.00 mm/s |
| Test speed | 0.50 mm/s | 0.50 mm/s |
| Post-test speed | 1.00 mm/s | 1.00 mm/s |
| Target mode | Force | 50% strain |
| Target force | 40.00 N | — |
| Trigger type | Auto (force) | Auto (force) |
| Trigger force | 0.50 N | 0.50 N |
| Load cell | 50 kg | 50 kg |

In addition, resilience testing was performed on each of the formulations initially in their unhydrated state, as well as after exposure for 1 hour to 0.1M HCl and PBS (pH 6.8) at 37±0.5° C. in accordance with the parameters for resilience testing (Table 2). Exposure to medium was accomplished by placing the multiparticulates in 50 mL PBS in glass reagent bottles of 100 mL capacity. The resilience of the matrix was calculated as the ratio of the AUC or work done by the multiparticulate on the probe after the maximum decompressive force was reached to the AUC or work done by the probe on the matrix up to the maximum compressive force (FIG. 3(b)).

Following generation of the polynomial equations relating the dependent and independent variables, the formulation process was optimized under constrained conditions for the measured responses DEE and MDT. Simultaneous equation solving for optimization of the formulation process was performed to obtain the levels of independent variables, which achieve the desired high drug entrapment and enteric-release characteristics (i.e. high DEE corresponding to increased drug loading and low MDT corresponding to slowest drug release achievable in acidic media).

The salting-out and cross-linking approach utilized yielded spherical enterosoluble matrices in a single processing step without the use of expensive machinery and organic solvents. Typical micrographs of synthesized formulations depict the variation in the morphology as a result of formulation variables (FIG. 4). High concentrations of plasticizer and with annealing of the multiparticulates at high temperatures (>42° C.) in addition to higher concentrations of the salting-out and cross-linking agent produced multiparticulates with a smooth surface and translucent appearance, observed for formulations 1, 2, 6, 8, 12, 14, 18, 19, 24, and 26 due to improved coalescence of the polymeric film. However, employing lower concentrations of the salting-out and cross-linking agent resulted in multiparticulates with surface precipitates, which were not adequately incorporated within the crosslinked matrix as observed for formulations 5, 7, 13, 17, 24, and 25. Low concentrations of TEC lead to the decreased degrees of polymeric plasticization as noted in formulations 11, 15, 21, 22, and 25.

The measured responses for the experimentally synthesized variants are shown in Tables 3 and 4. The aspect ratio suggests multiparticulates of near-spherical geometry (AR=1) with little variation in the particle diameter within each formulation (monodisperse) indicative of good flow properties, and no statistically significant variation in size between formulations (P>0.05). Complexometric determination of Zn²⁺ revealed that 23.70 to 287.89 moles zinc per mole of methacrylic acid copolymer was implicated in cross-link formation. Drug content ranged from 4.74-13.88 mg per 100 mg of multiparticulates. Entrapment efficiencies of 27.92% to 99.77% were obtained. The ability of the multiparticulates to retard drug release in acidic media varied greatly—drug release at t₂ₕ ranged from 1.67% to 73.04% (FIG. 5). Drug release in alkaline media was comparatively rapid for all variants owing to hydration of the carboxylic acid groups of the methacrylic acid copolymer with the resultant dissolution of the multiparticulate matrix. Polymeric disintegration in alkaline media may also be promoted by removal of Zn²⁺ from the matrix due to the sequestration of these cations by phosphate ions within the buffer solution to form insoluble chelates. Similar sequestration by complexing ions would be expected to occur during intestinal transit.¹⁸

The first approach for multi-step crosslinking of the polymethacrylates revealed a drug loading of 32%, 30% and 12%% for the ZnSO₄/MgSO₄ combination multiparticulates, ZnSO₄ multiparticulates and for the ZnSO₄ cured in MgSO₄ respectively. Dissolution profiles displayed 33%, 44% and 53% of drug release after 9 hours for the ZnSO₄ cured in MgSO₄ multiparticulates, ZnSO₄/MgSO₄ combination multiparticulates and the ZnSO₄ multiparticulates respectively.

Results attained for the multiparticulates generated employing the second multi-step approach revealed a drug loading of 45-61% w/w. Dissolution profiles displayed desirable controlled drug release of 100% in 12 hours.

The physical and mechanical properties of polymers are extensively influenced by the chemical composition of the polymer such as the degree of cross-linking and the type and quantity of plasticizer employed.² The textural behaviour of the multiparticulate formulations (Table 4) highlights the variation in their physicomechanical properties. This variation was not statistically significant (P>0.05), however trends could be identified.

TABLE 3
Measured responses for the multiparliculate formulations*

| | | | | Fractional | | |
|---|---|---|---|---|---|---|
| | | Drug | | Drug | | |
| Experimental | n_{Zn} | Content | DEE | Release | MDT | Aspect |
| Formulation | (mol) | (mg/100 mg) | (%) | (t_{Zh}) | (t_{Zh}) | Ratio |
| 1 | 89.72 | 9.51 | 92.01 | 0.218 | 0.169 | 1.22 |
| 2 | 71.53 | 9.48 | 56.79 | 0.224 | 0.202 | 1.02 |
| 3 | 136.11 | 7.02 | 50.47 | 0.208 | 0.101 | 1.16 |
| 4 | 154.17 | 7.39 | 46.19 | 0.211 | 0.134 | 1.15 |
| 5 | 145.83 | 6.96 | 40.53 | 0.699 | 0.344 | 1.03 |
| 6 | 28.33 | 13.88 | 88.37 | 0.148 | 0.113 | 1.12 |
| 7 | 115.28 | 5.31 | 30.84 | 0.510 | 0.291 | 1.10 |
| 8 | 64.86 | 9.87 | 67.41 | 0.141 | 0.123 | 1.08 |
| 9 | 140.83 | 6.83 | 45.20 | 0.091 | 0.059 | 1.11 |
| 10 | 178.61 | 6.69 | 47.21 | 0.238 | 0.125 | 1.11 |
| 11 | 203.88 | 6.68 | 42.39 | 0.230 | 0.141 | 1.21 |
| 12 | 23.611 | 12.09 | 80.57 | 0.250 | 0.221 | 1.07 |
| 13 | 181.38 | 7.97 | 52.11 | 0.730 | 0.379 | 1.17 |
| 14 | 75.55 | 7.59 | 47.59 | 0.103 | 0.076 | 1.07 |
| 15 | 205.55 | 5.68 | 35.73 | 0.235 | 0.164 | 1.14 |
| 16 | 47.91 | 9.74 | 67.99 | 0.151 | 0.135 | 0.99 |
| 17 | 130.83 | 7.16 | 39.47 | 0.583 | 0.312 | 1.01 |
| 18 | 143.88 | 6.91 | 43.02 | 0.095 | 0.047 | 1.10 |
| 19 | 72.36 | 10.27 | 62.68 | 0.101 | 0.087 | 1.13 |
| 20 | 154.17 | 7.27 | 46.61 | 0.177 | 0.096 | 1.15 |
| 21 | 229.44 | 4.74 | 27.92 | 0.236 | 0.141 | 1.10 |
| 22 | 287.78 | 7.43 | 54.39 | 0.017 | 0.009 | 1.20 |
| 23 | 203.19 | 6.02 | 39.32 | 0.387 | 0.195 | 1.11 |
| 24 | 147.60 | 7.41 | 44.54 | 0.662 | 0.337 | 1.18 |
| 25 | 155.69 | 6.52 | 39.24 | 0.708 | 0.364 | 1.24 |
| 26 | 36.39 | 11.50 | 99.77 | 0.205 | 0.171 | 1.33 |

(*Results are expressed as mean of at least 3 measurements, for Aspect Ratio n = 50. S.D.s were less than: n_{Zn} ± 2.50, Drug content ± 0.93, DEE ± 3.79, Drug release ± 0.034, MDT ± 0.012)

TABLE 4
Measured textural properties of the
experimentally synthesized variants**

| | | Mean HCl | Mean PBS | Defor- | |
|---|---|---|---|---|---|
| | Mean Dry | hydrated | hydrated | mation | Matrix |
| Experimental | Resilience | resilience | resilience | Energy | Hardness |
| Formulation | (%) | (%) | (%) | (J × 10²) | (N/mm) |
| 1 | 6.80 | 6.19 | 5.42 | 1.20 | 38.10 |
| 2 | 14.69 | 5.40 | 5.03 | 1.75 | 44.11 |
| 3 | 9.43 | 17.57 | 15.52 | 0.40 | 175.62 |
| 4 | 7.91 | 9.14 | 6.25 | 0.40 | 209.86 |
| 5 | 6.29 | 6.02 | 6.33 | 0.97 | 21.16 |
| 6 | 14.46 | 5.75 | 6.95 | 2.15 | 30.83 |
| 7 | 5.97 | 6.40 | 6.10 | 0.67 | 186.88 |
| 8 | 13.24 | 5.75 | 6.51 | 0.73 | 21.73 |
| 9 | 6.40 | 9.03 | 11.33 | 0.45 | 157.47 |
| 10 | 5.25 | 13.58 | 8.84 | 0.40 | 194.14 |
| 11 | 9.19 | 9.55 | 10.30 | 0.70 | 18.91 |
| 12 | 8.95 | 9.71 | 9.64 | 1.35 | 57.48 |
| 13 | 4.38 | 7.74 | 5.95 | 0.80 | 23.36 |
| 14 | 2.36 | 6.01 | 6.73 | 0.50 | 23.89 |
| 15 | 11.41 | 12.25 | 5.10 | 1.05 | 20.90 |
| 16 | 14.19 | 6.54 | 6.67 | 1.30 | 46.11 |
| 17 | 6.94 | 3.78 | 5.12 | 0.53 | 21.47 |
| 18 | 2.93 | 11.60 | 9.12 | 1.63 | 27.02 |
| 19 | 6.88 | 5.10 | 7.04 | 0.87 | 24.22 |
| 20 | 8.77 | 9.14 | 6.25 | 0.40 | 180.48 |
| 21 | 10.27 | 5.26 | 3.75 | 0.63 | 169.27 |
| 22 | 4.62 | 14.94 | 6.44 | 0.40 | 202.14 |
| 23 | 11.96 | 17.49 | 10.72 | 0.40 | 201.31 |
| 24 | 1.05 | 15.61 | 8.85 | 1.13 | 31.33 |
| 25 | 6.77 | 11.15 | 8.10 | 0.55 | 26.63 |
| 26 | 5.79 | 6.03 | 6.47 | 1.30 | 27.73 |

(**Results are expressed as the mean of at least 3 measurements, S.D.s obtained were less than: Resilience ± 0.16, Deformation Energy ± 4.18 × 10⁻⁴, Matrix Hardness ± 4.37)

Resilience is defined as the ability of a strained body to recover its size end shape after deformation caused especially by compressive stress, a concept derived from the Huber-Hencky Theory of Strength.¹⁹ The resilience of formulations 3, 4, 9, 10, 11, 12, 13, 14, 15, 18, 20, 22, 24, 24, 26 improved by 0.36 to 14.56% with hydration suggestive of enhanced control over drug release, whereas the resilience of the other formulations was reduced (0.27 to 5.01%) following exposure to dissolution media (FIG. 8).

More important is the relationship between the acid-hydrated resilience and drug release in acidic media (FIG. 8). In general, as the resilience on hydration in acidic media increased (28%), the extent of control over drug release increased (<30% drug release at t₂ₕ). The increase in hydration may serve to reinforce the matrix of these variants. However, more extensive hydration resulted in the formation of a loose gel matrix in certain formulations and a lower resilience. This trend was only observed for 61.54% of the formulations and the acid-hydrated resilience of the multiparticulates could not always serve as a significant predictor of drug release.

The matrix hardness of the multiparticulates was generally greater when intermediate to low concentrations of plasticizer were incorporated, due to less softening of the polymeric matrix; whereas the energy required to rupture the multiparticulate matrices was greater in formulations incorporating high plasticizer concentrations, as an increased degree of plasticization decreased brittleness and improved the flexibility and distensibility of the polymeric chains which led to the dissipation of larger amounts of energy when exposed to shear forces.^{2,3} An increased degree of cross-linking would also be expected to improve the mechanical hardness of the polymer matrix.²⁰ In formulations that exhibited a high cross-link density due to a high n_{Zn}, the chain segments between the cross-links were short and anchored by many points, causing a loss in flexibility and an increase in matrix rigidity. This correlational behavior between n_{Zn} (as a measure of the cross-link density) and the matrix hardness was demonstrated in the multiparticulate formulations (FIG. 8) by a dramatic increase in matrix hardness at high levels of Zn²⁺ (>150 mol).

The n_{Zn}, DEE and MDT for the experimentally synthesized formulations were included in the statistical design for identification of a formulation with an optimal drug entrapment and dissolution profile in acidic media.

Residual analysis (run order, predicted values) for the n_{Zn}, DEE and MDT data (FIG. 9) generally showed random scatter i.e. no trends, indicating none of the underlying assumptions of the multiple regression analysis were grossly violated; however some fanning and an outlier was observed for n_{Zn} (FIG. 9(a) indicative of a degree of non-constant variance. The normal probability plots of the residuals fell on a straight line indicating the data to be normally distributed with no evidence of unidentified variables.

The residuals and standardized residuals indicated that most cases were adequately fitted by the response surface model. Cook's distance is an overall measure of the combined impact of each observation on the fitted values and considers whether an observation is unusual with respect to both x- and y-values. Unusual observations generated by the model were minimal. The significance of the ratio of mean square variation due to regression and residual error was tested using ANOVA. The theoretical (predicted) values and observed (experimental) values were in close agreement as seen from Table 6 for n_{Zn} (R²=93.42%), DEE (R²=93.73%) and MDT (R²=95.12%) respectively, thus indicating the applicability of the regression models and usefulness of response surface plots.

The Pearson correlation coefficient (R and R-adjusted) represents the proportion of variation in the response that is explained by the model. The R² (87.3%, 87.9%, 90.5%) and R²-adjusted (71.1%, 72.4%, 78.3%) values for the nZn, DEE and MDT model were satisfactory.

The significance of linear and higher-order interaction terms is depicted by the p-values in Table 5. The salting-out and cross-linking agent significantly affected n_{Zn} (p=0.034) and the DEE (p=0.000), as did the plasticizer concentration employed (p=0.000 and p=0.002 respectively). High drying temperatures (≧42.5° C.) also significantly improved DEE (p=0.029). ZnSO₄ had a significant effect on the MDT (p=0.000). A significant interaction effect was observed between ZnSO₄ and TEC variables on n_{Zn} (p=0.005) and on drug release in acidic media (p=0.035).

TABLE 5
Estimated p-values for the measured responses

| | p-value | | |
|---|---|---|---|
| Term | n_{zn} | DEE | MDT |
| ZnSO₄ | 0.034 | 0.000 | 0.000 |
| CRT | 0.955 | 0.271 | 0.925 |
| DT | 0.839 | 0.029 | 0.055 |
| TEC | 0.000 | 0.002 | 0.613 |
| ZnSO₄ * ZnSO₄ | 0.167 | 0.044 | 0.000 |
| CRT * CRT | 0.312 | 0.583 | 0.766 |
| DT * DT | 0.661 | 0.790 | 0.848 |
| TEC * TEC | 0.871 | 0.931 | 0.297 |
| ZnSO₄ * CRT | 0.586 | 0.122 | 0.502 |
| ZnSO₄ * DT | 0.353 | 0.164 | 0.668 |
| ZnSO₄ * TEC | 0.005 | 0.235 | 0.035 |
| CRT * DT | 0.540 | 0.789 | 0.908 |
| CRT * TEC | 0.546 | 0.080 | 0.314 |
| DT * TEC | 0.985 | 0.658 | 0.965 |

The complete regression equations generated for n_{Zn}, DEE and MDT are indicated below:

n_{Zn}=22.738+4.375[ZnSO₄]+4.032[CRT]+1.839[DT]+7.551[TEC]−0.0638[ZnSO₄*ZnSO₄]−0.036[CRT*CRT]−0.025[DT*DT]+0.179[TEC*TEC]+0.022[ZnSO₄*CRT]+0.050[ZnSO₄*DT]−0.795[ZnSO₄*TEC]−0.029[CRT*DT]−0.125[CRT*TEC]0.005[DT*TEC] [4]

DEE=83.487−1.002[ZnSO₄]−1.588[CRT]−0.073[DT]−2.289[TEC]+0.027[ZnSO₄* ZnSO₄]+0.005[CRT*CRT]+0.004[DT*DT]+0.026[TEC*TEC]+0.018[ZnSO₄*CRT]−0.021[ZnSO₄*DT]+0.078[ZnSO₄*TEC]+0.003[CRT*DT]+0.106[CRT*TEC]−0.032[DT*TEC] [5]

MDT=0.657−0.027[ZnSO₄]+1.873E−03[CRT]+8.209E−04[DT]−0.028[TEC]+2.856E−04[ZnSO₄* ZnSO₄]−1.391E−05[CRT*CRT]−1.479E−05[DT*DT]+1.583E−03[TEC*TEC]+3.722E−05[ZnSO₄*CRT]−3.036E−05[ZnSO₄*DT]+7.256E−04[ZnSO₄*TEC]−7.238E−06[CRT*DT]−2.831E−04[CRT*TEC]−1.536E−05[DT*TEC [6]

TABLE 6
Correlation between experimental and predicted values for n_{Zn}, DEE, and MDT

| n_{Zn} (mol) | | | DEE (%) | | | | |
|---|---|---|---|---|---|---|---|
| | | Cook's | | | Cook's | MDT | |
| Experimental | Predicted | Distance | Experimental | Predicted | Distance | Experimental | Predicted |
| 89.72 | 95.97 | 0.007 | 92.01 | 88.98 | 0.021 | 0.169 | 0.134 |
| 71.53 | 75.01 | 0.002 | 56.79 | 48.53 | 0.156 | 0.202 | 0.167 |
| 136.11 | 114.00 | 0.085 | 50.47 | 55.58 | 0.060 | 0.101 | 0.132 |
| 154.17 | 154.17 | 0.000 | 46.19 | 46.40 | 0.000 | 0.134 | 0.115 |
| 145.83 | 145.85 | 0.000 | 40.53 | 45.76 | 0.062 | 0.344 | 0.308 |
| 28.33 | 53.79 | 0.112 | 88.37 | 74.28 | 0.453 | 0.113 | 0.114 |
| 115.28 | 126.06 | 0.020 | 30.84 | 37.34 | 0.097 | 0.291 | 0.308 |
| 64.86 | 74.55 | 0.016 | 67.41 | 65.73 | 0.006 | 0.123 | 0.103 |
| 140.83 | 119.53 | 0.078 | 45.20 | 47.89 | 0.017 | 0.059 | 0.069 |
| 178.61 | 137.98 | 0.286 | 47.21 | 59.49 | 0.344 | 0.125 | 0.135 |
| 203.89 | 201.38 | 0.001 | 42.39 | 43.84 | 0.005 | 0.141 | 0.102 |
| 23.61 | −31.60 | 0.527 | 80.57 | 93.22 | 0.365 | 0.221 | 0.216 |
| 181.39 | 161.33 | 0.070 | 52.11 | 40.73 | 0.295 | 0.379 | 0.348 |
| 75.56 | 77.38 | 0.001 | 47.59 | 50.61 | 0.021 | 0.077 | 0.112 |
| 205.56 | 225.01 | 0.065 | 35.73 | 31.36 | 0.044 | 0.154 | 0.150 |
| 47.92 | 72.36 | 0.103 | 68.00 | 69.44 | 0.005 | 0.135 | 0.174 |
| 130.83 | 145.06 | 0.035 | 39.47 | 47.33 | 0.141 | 0.312 | 0.344 |
| 143.89 | 141.11 | 0.001 | 43.02 | 38.54 | 0.046 | 0.047 | 0.076 |
| 72.36 | 115.36 | 0.320 | 62.68 | 61.43 | 0.004 | 0.087 | 0.080 |
| 154.17 | 154.17 | 0.000 | 46.61 | 46.40 | 0.000 | 0.096 | 0.115 |
| 229.44 | 225.48 | 0.003 | 27.92 | 31.30 | 0.026 | 0.141 | 0.100 |
| 287.78 | 244.35 | 0.326 | 54.40 | 56.97 | 0.015 | 0.009 | 0.085 |
| 203.19 | 221.85 | 0.060 | 39.32 | 41.12 | 0.007 | 0.196 | 0.157 |
| 147.50 | 130.73 | 0.049 | 44.54 | 41.17 | 0.026 | 0.337 | 0.309 |
| 155.69 | 167.49 | 0.024 | 39.24 | 34.40 | 0.054 | 0.364 | 0.409 |
| 36.39 | 76.10 | 0.273 | 99.77 | 90.51 | 0.196 | 0.171 | 0.161 |

Main effects, interaction and response surface plots were obtained for the measured responses (n_{Zn}, DEE, MDT) based on the experimental model. The relationship between the independent variables and the responses can be further explained through graphical illustration of the effect of the independent variables and their interactions. The interaction effects are estimated by subtracting the mean positive response values from the mean negative response values. The estimated interaction effects of the responses studied are shown in FIG. 10. The main effects plots (FIG. 11) are used in conjunction with the ANOVA for the determination of the strength or relative significance of the effects across factors. The surface plots generated (FIG. 12) represent the functional relationship between the response and the experimental factors.

Increasing the degree of plasticization (high TEC levels) had a significant negative effect on n_{Zn}, incorporated within the crosslinked matrix (p=0.000). The increase in polymer chain mobility afforded by the addition of a plasticizer is necessary for adequate coalescence of the polymeric film, however, high degrees of plasticization may negate the favorable chain alignment required for incorporation of Zn²⁺ within the ionic cross-link between the polymer's carboxylic acid side chains. This is as a result of the plasticizer's ability to weaken polymeric intermolecular attractions and to increase the polymer's free volume. An increase in ZnSO₄ in the salting-out and cross-linking solution (≧30% w/v) also had a significant negative effect on the amount of Zn²⁺ incorporated (P=0.034). As the aqueous dispersion is extruded into the salt solution, the droplet surface immediately encounters high concentrations of Zn²⁺ inducing film formation. This may hinder more significant penetration of the cation into the internal matrix for participation in cross-linking. The interaction between these two variables also proved to have significant opposing effects on the molar amount of Zn²⁺ implicated in cross-link formation within the internal matrix (p=0.005). n_{Zn} was maximal at either high TEC levels and low ZnSO₄ levels and vice versa as depicted in FIG. 10(a).

The effect of factors ZnSO₄ and DT at the midpoint of factors TEC and CRT on n_{Zn} is shown in FIG. 12(a). As the concentration of the salting-out solution increases, there is an observed decrease in n_{Zn}. This may be due to the rapidly coalesced polymer film hindering a more notable penetration of the Zn²⁺ into the internal matrix.

The effect of factors ZnSO₄ and TEC at the midpoint of factors CRT and DT on the response n_{Zn} is shown in FIG. 12(b). At low levels of ZnSO₄, an increase in TEC caused an increase in n_{Zn}, however, at high levels of ZnSO₄ there is a significant decrease in n_{Zn} as TEC increases from 2 to 10% w/w. The optimal polymer chain alignment for ionic cross-linking with Zn²⁺ thus occurs at opposing levels of ZnSO₄ and TEC.

Inspection of the interaction and main effects plots generated (FIGS. 10(b) and 11(b)) illustrate the significance of the effect of the concentration of the salting-out and cross-linking agent and the plasticizer on the effective amount of drug entrapped within the multiparticulate matrices. Factors promoting cross-link formation and polymer coalescence facilitate the formation of a dense matrix which retards drug release to a greater extent.^{21,22,23} The increased availability of the Zn²⁺ in the salting-out and cross-linking solution at higher concentrations promotes gel shrinkage and the formation of intra- and intermolecular ionic cross-links within and between the polymer chains, producing a dense, interconnected enteric film in which drug entrapment is more likely and which retains its integrity in acidic dissolution media, slowing the release of isoniazid through the reduced interstices of the multiparticulate. The drug entrapment was increased with an increase in TEC. Because matrix formation is considered to be dependent on the concentration of incorporated plasticizer, the coalescence of the polymer particles is likely to be enhanced by increasing TEC, with improved entrapment of isoniazid. High DT values also significantly improved DEE (p=0.029). The reduced porosity and enhanced coalescence of multiparticulates dried at temperatures ≧42.5° C. used in this study resulted in a decrease in drug leaching to the enteric film surface. Coalescence of particles within the multiparticulates is improved when the drying temperature is set close to the MFT of the polymer-plasticizer systems comprising the multiparticulates.

The effect of factors DT and TEC at the midpoint of factors ZnSO₄ and CRT on response DEE is shown in FIG. 12(c). At low levels of factor TEC, the DEE is low and increasing factor DT further lowers the DEE. At high levels of TEC, the DEE improves, however, an increase in the DT factor still results in a reduction in the DEE due to leaching of the drug out of the highly plasticized, pliable structure when exposed to temperatures ≧42.5° C. The DEE is maximal at a low DT factor level and at high TEC levels.

The effect of factors ZnSO₄ and TEC at the midpoint of factors CRT and DT on the response DEE is shown in FIG. 12(d). At low levels of factor TEC, an increase in ZnSO₄ causes an increase in the DEE. High levels of TEC result in a significant improvement in the DEE as factor ZnSO₄ increases from 10 to 50% w/v. The increase in polymeric chain mobility afforded by increased degrees of plasticization could aid in rapidly orientating the polymeric chains for formation of a coalesced matrix, which facilitates isoniazid entrapment within its network structure.

A significant interaction effect was observed between ZnSO₄ and TEC variables (P=0.035), and drug release in acidic media was minimal when a high concentration of ZnSO₄, in combination with a low concentration of TEC was employed in the formulation of multiparticulates. Increasing the concentration of ZnSO₄ from 10 to 30% w/v had a significant negative effect on the MDT (p=0.000). An increase in the availability of Zn²⁺ in the salting-out and cross-linking solution promotes the formation of a crosslinked spherical enteric film of improved gastro-resistance. Cross-linking of the internal matrix is also promoted, except at very high concentrations of the salting-out and cross-linking solution (>30% w/v). The formation of cross-links between polymer chains ultimately forms a dense, interconnected polymeric film and internal matrix which retains its integrity in acidic dissolution media, slowing the diffusion of isoniazid through the reduced interstices of the polymeric structure (see FIG. 13(a) (b) and (c)). The phenomenon of enhanced salting-out and cross-linking of the enteric copolymer in the presence of a higher concentration of cation is described by the Schulze-Hardy rule, which governs the ability of an electrolyte to reduce the value of the zeta-potential of the polymer.

An increase in the drying temperature from 42.5 to 60° C. caused an overall reduction in the amount of drug released in acidic media. The reduced porosity and enhanced coalescence of multiparticulates dried at temperatures 42.5° C. resulted in prolongation of the total release time and a decrease in the MDT. The MDT is minimal at high levels of both ZnSO₄ and DT. Sufficient annealing of the multiparticulate (at temperatures ≧42.5° C.) softens the polymer causing it to fill the interstices and resulting in the observed morphological changes. Thus, the reduced porosity and enhanced coalescence of multiparticulates dried at these elevated temperatures (≧42.5° C.) probably resulted in a decrease in the release rate and a prolongation of the total release time. The drying temperature employed may thus be related to the T_{g} and MFT of the polymer-plasticizer systems constituting the multiparticulates. Coalescence of particles within the polymeric matrix is improved when the drying temperature is set close to the MFT of the polymer-plasticizer systems.

Plasticizers are added to film forming polymers to modify physical properties of the polymers and to improve their film forming characteristics as well as their permeability, hence controlling the drug release ^{22,23}. Though the plasticizers included in the polymeric dispersion serve to decrease the MFT and T_{g}, they also increase the free volume in the polymeric matrix, which in turn facilitates the release of drug from the multiparticulate.^{24,25} A significant interaction effect was observed between ZnSO₄ and TEC variables (p=0.035), and a low MDT was observed when a high concentration of ZnSO₄, in combination with a low concentration of TEC was employed in the formulation of the multiparticulates. The effect of factors ZnSO₄ and TEC and their interaction at the midpoint of factors DT and CRT on response MDT is shown in FIG. 12(e). At low levels of TEC, an increase in ZnSO₄ from 10 to 50% w/v results in a significant decrease in MDT. Although high levels of plasticizer improve polymer coalescence, the free volume of the polymer is increased, with a resultant increase in drug release as discussed above.

The effect of factors DT and TEC at the midpoint of factors ZnSO₄ and CRT on response MDT is shown in FIG. 12(f). At high and low levels of TEC, an increase in DT significantly reduces MDT. Increasing the TEC concentration employed in the formulation of the multiparticulates does not significantly slow the rate at which drug diffuses out of the polymeric matrix, even when elevated drying temperatures (≧42.5° C.) are employed.

Response optimization procedure¹⁴ (MINITAB®, V14, Minitab, USA) was used to obtain the optimized levels of ZnSO₄, CRT, DT and TEC. Three optimal formulations were developed following constrained optimization of DEE, constrained optimization of MDT and simultaneous constrained optimization of DEE and MDT (F1, F2, and F3). An MDT value representing controlled release in acidic media such that ≦3% of the entrapped drug would be released during the first and second hour respectively was targeted (MDT≦0.07). This was in order to ensure drug release in accordance with the USP 24 specifications for drug release from enteric-release articles (<5% at t₁ₕ and <10% at t₂ₕ). The optimized levels of the independent variables that would achieve the desired dissolution and entrapment properties and their predicted responses were then determined.

The optimized levels of the independent variables, the goal for the response, the predicted response, y, at the current factor settings, as well as the individual and composite desirability scores are shown in FIG. 14. Based on the statistical desirability function, it was found that the composite desirabilities for each of the formulations was >0.9. The constrained settings utilized are outlined in Table 7.

TABLE 7
Constrained settings for response optimization

| Parameters | Constraint |
|---|---|
| ZnSO₄ | 10-50% ^{w}/ᵥ |
| CRT | 15-60 minutes |
| DT | 25-60° C. |
| TEC | 2-10% ^{v}/ᵥ |
| DEE | 80-90% |
| MDT | 0.05-0.07 |

The ideal formulations were prepared according to the optimal predicted settings. The experimentally derived values for the DEE and MDT of the optimal formulations were in close agreement with the predicted values (Table 8), demonstrating the reliability of the optimization procedure in predicting the dissolution behaviour of the novel salted-out enteric-release systems and ascertaining the significance of the effect of ZnSO₄, DT and TEC on isoniazid entrapment and release from the multiparticulates.

TTABLE 8
Experimental and Predicted Response Values for the Optimized
Formulations

| Measured | | | | |
|---|---|---|---|---|
| Response | Formulation | Predicted | Experimental | Desirability |
| DEE (%) | F1 | 90.000 | 91.066 | 1.000 |
| | F2 | ^{a} | 47.414 | ^{a} |
| | F3 | 76.731 | 72.515 | 0.837 |
| MDT | F1 | ^{b} | 0.107 | ^{b} |
| | F2 | 0.050 | 0.066 | 1.000 |
| | F3 | 0.070 | 0.069 | 1.000 |

^{a}Formulation optimized for DEE only
^{b}Formulation optimized for MDT only

Simultaneous optimization of DEE and MDT resulted in the fabrication of an optimum gastro-resistant system (F3) with adequate drug entrapment. The dissolution profile of the optimum multiparticulate formulation is depicted in FIG. 15. Drug release was sufficiently retarded in accordance with the USP specifications for enteric-release articles; however, it needs to be ascertained whether significant segregation of RIF and INH is attained upon co-administration, with INH delivered as multiparticulates. High performance liquid chromatographic (HPLC) analysis of in vitro dissolution samples following drug release testing of a RIF-INH fixed dose combination as reported by Mohan et al. and Prabakaran et al. needs to be undertaken for thorough evaluation of the benefits of the enteric-release system in this regard. The complete HPLC assay, being beyond the scope of the experimental design strategy reported here, will be delineated in future studies.

It is envisaged that the above-described process may be scaled-up for large scale or industrial manufacture of multiparticulates. This may be achieved in a spray-drying apparatus. The variety of atomisation, drying and separation techniques enables spray-drying to be adapted to many applications, including low-temperature spray-drying and spray polycondensation.²⁶

The set-up and process staging is schematically illustrated in FIG. 16. The electrolyte solution, at the optimum concentration setting, is sprayed into the drying chamber of the spray dryer maintained at a relatively low temperature for the aqueous dispersion feed, saturating the drying chamber with the salting-out and cross-linking electrolyte. The drug-loaded aqueous dispersion is pumped at a controlled rate into the spray-dryer where it is atomised into droplets using a rotary atomiser. The atomiser facilitates the production of near-uniform droplets that is contacted by electrolyte-saturated air-filled chamber maintained at the designated temperature setting for optimum annealing of the plasticized multiparticulate matrix. This induces the formation of an unmitigated enteric film and an internal matrix exhibiting the optimum degree of cross-linkage.

The hot air eventually evaporates the water from the formed multiparticulate, leading to ultimate solidification. The solid multiparticulates exit the spray-dryer in a gas stream and are separated in the product collection operation.

A fixed-dose combination incorporating INH-loaded multiparticulates and RIF in a suitable instant-release form would facilitate differentiated delivery of RIF and INH in the gastrointestinal tract. Considering the final dosage form, the multiparticulates could be filled into hard gelatin capsules or compressed into tablets together with RIF. Formulation of these controlled release multiparticulates into these conventional dosage forms may result in several problems being encountered. Risk of tampering has somewhat reduced the use of hard gelatin capsules.

For successful compression of multiparticulates, good flow properties are essential and the polymeric coating must be capable of resisting severe mechanical stress during compression. Poor flow properties result in content uniformity problems. Compression under high pressure can cause multiparticulates to rupture with loss of controlled release action. Poor compressibility of the multiparticulates often requires the addition of large amounts of easily compressible excipients. This dilution effect could thus result in a too-low drug content in the final dosage form, which would be undesirable in this system where a large dose of the anti-TB agents need to be delivered efficiently to the patient.²⁷ Fassihi²⁸ has studied the consolidation behaviour of polymeric particles. Plastic deformation and particle fusion were reported to be in operation during compression. Thus, the possible fusion that could occur during compression results in a disintegrating matrix with loss of character of the multiparticulate dosage form and a possible reduction in drug release.

An alternative approach for the oral administration of multiparticulates is to suspend them in a liquid vehicle to form a suspension dosage form or incorporate them into a dry powder system, which is to be reconstituted with water by the patient immediately prior to administration. In addition to overcoming the aforementioned obstacles to the delivery of multiparticulates, they also provide the patient with ease of swallowing and dosing flexibility and are thus preferred among certain patient groups such as infants, children and the elderly.²⁷

Problems such sedimentation and caking of the suspended particles or degradation of the RIF or INH, leaching of the INH from the suspended multiparticulates into the carrier vehicle during storage, alterations in the enteric-release pattern of the multiparticulates because of interactions between the vehicle and the coating material, are overcome with reconstitutable multiparticulates that are dispersed in a liquid vehicle just prior to use.²⁹

It was rationalized that a RIF-INH anti-TB combination be administered to the patient as a dry dispersible multiparticulate system containing the modified-release multiparticulates. The dry system incorporates RIF and appropriate suspending and gel-forming polymers, for reconstitution in water immediately prior to administration to the patient, and disperses rapidly in water to form a three-dimensional supportive network for facilitated multiparticulate delivery.

The dry system, formulated as reconstitutable granules, incorporates at least one hydrophilic gel-forming viscosity-enhancing agent for adequate suspension of the INH-loaded multiparticulates and the incorporated RIF. The appropriate gel-forming agent/s, selected from pharmaceutically acceptable viscosity agents, which includes, xanthan gum, hydroxypropylmethyl cellulose, methylcellulose, carageenan, carboxymethyl cellulose, microcrystalline cellulose, polyvinylpyrrolidone, soluble starches and carbomers, are required to disperse and gel rapidly to form a suspension possessing the necessary properties for extemporaneous use. Previous work³⁰ confirmed that a hydrophilic polymeric composite system comprising two suspending and gel-forming agents that includes a combination of a polysaccharide gums such as xanthan, guar gum, or carrageenan and a soluble starch-based system was most effective. The soluble starches employed (e.g. pregelatinised starch or sodium starch glycolate) in the gel-forming suspension system demonstrate dual functionality as a hydrophilic suspending agent and granule disintegrant.

### CONCLUSION

In this study, the Box-Behnken Response Surface Design found application in the development and optimization of a novel approach for the fabrication of ionotropically salted-out and crosslinked multiparticulates for delivery of isoniazid to the small intestine. The design generated a range of spherical formulations, which varied in their resilient nature, matrix hardness, deformation energy, and drug entrapment and release characteristics. The use of RSM proved to be a compelling option for the identification of critical and significant formulation variables and processing variables such as ZnSO₄, TEC and DT. The salting-out and cross-linking agent and plasticizer significantly affected n_{Zn} and the DEE. The temperature at which the multiparticulates were annealed also significantly affected the DEE. ZnSO₄ and the interaction between ZnSO₄ and TEC had a significant effect on the MDT.

Regression analysis demonstrated the agreement between the predicted and observed responses obtained, indicating the applicability of the models generated by the Box-Behnken design. Additional experiments performed at the optimal variable settings confirmed the validity and reliability of the proposed models in predicting the drug entrapment and dissolution behaviour of the salted-out enteric-release systems.

Simultaneous optimization of the formulation and processing variables resulted in the fabrication of an optimal formulation having a DEE of 72.51% and a MDT of 0.069, which was capable of adequately controlling isoniazid release in acidic media and releasing >90% of the drug after 2 hours at intestinal pH. The fabrication of an optimal crosslinked multiparticulate system in a single processing step is thus a satisfactory alternative to the standard technique for manufacturing enteric-release multiparticulates.

Spray-drying apparatus was successfully implemented for large-scale manufacture of multiparticulates according to the described process.

A suitable means for delivery of the multiparticulate system as dispersible multiparticulates for reconstitution immediately prior to administration to the patient was also proposed.

It is envisaged that the above-described invention is able to bypass difficulties associated with multiple dosing of active pharmaceutical compositions with intolerable side effects and providing a pharmaceutical dosage form that is able to avoid possible deleterious interactions amongst at least two or more incompatible active pharmaceutical compositions whilst incorporated into the said pharmaceutical dosage form as a single or plurality of a heterogeneously configured multiparticulate system for the gastrointestinal delivery in a human or animal body. It is also envisaged that crosslinking improves the physicochemical and physicomechanical properties of the multiparticulates and the ability to modulate drug release.

The invention also provides a means of crosslinking so as to improve the physicochemical and physicomechanical properties of the multiparticulates and the ability to modulate drug release and an approach for improving the drug entrapment efficiency of the various multiparticulate systems developed.

This work was supported by a National Research Foundation Scarce Skills Scholarship (South Africa) and a grant awarded by the Medical Faculty Research Endowment Fund (University of the Witwatersrand, Johannesburg, South Africa).

### REFERENCES

1. Allain C and Salome L. 1990. Gelation of semidilute polymer solutions by ion-complexation: Critical behavior of the rheological properties versus cross-linking. Macromolecules 23:981-987.
2. Nielson L E and Landel R F. 1993. Mechanical Properties of Polymers and Composites, Vol. 2, New York: Marcel Dekker, p. 15-22.
3. McGinity J W (editor). 1996. Aqueous Polymeric Coatings for Pharmaceutical Dosage Forms, New York: Marcel Dekker, p. 62-75, 108.
4. Sastry S V, Reddy I K and Khan M A. 1997. Atenolol gastrointestinal therapeutic system: optimization of formulation variables using response surface methodology. J Contr Rel 45(2):121-130.
5. Guo H X, Heinamaki J, Yliruusi J. 2002. Diffusion of a freely water-soluble drug in aqueous enteric-coated pellets. AAPS PharmSciTech 3(2): article 16.
6. Bianchini R, Resciniti M, Vecchio C. 1991. Technology evaluation of aqueous enteric coating systems with and without insoluble additives. Drug Dev Ind Pharm 17:1779-1794.
7. Eby G. 2005. Preventing Anosmia from Intranasal Zinc Administration. http://www.coldcure.com/anosmia/anosmia.html
8. Cohen S, Andrianov A K, Wheatley M, Allcock H R, Langer R S. 1996. Gas-filled polymeric microbubbles for ultrasound imaging. U.S. Pat. No. 5,487,390.
9. Chambers H F. 2001. Antimycobacterial drugs. In Katzung, B G, editor. Basic and clinical pharmacology, 8^{th} ed., New York: McGraw-Hill. p. 803-318.
10. Mohan B, Sharda N, and Singh S. 2003. Evaluation of the recently reported USP gradient HPLC method for analysis of anti-tuberculosis drugs for its ability to resolve degradation products of rifampicin. J Pharm Biomed Anal 31(3): 607-612.
11. Sankar R, Sharda N, Singh S. 2003. Behaviour of decomposition of rifampicin in the presence of isoniazid on the pH range 1-3. Drug Dev Ind Pharm 29(7):733-8.
12. Mariappan T T, Jindal K C and Singh S. 2004. Overestimation of rifampicin during colorimetric analysis of anti-tuberculosis products containing isoniazid due to formation of isonicotinyl hydrazone. J Pharm Biomed Anal. 36(4):905-908.
13. Panchagnula R and Agrawal S. 2004. Biopharmaceutic and pharmacokinetic aspects of variable bioavailability of rifampicin. Int J Pharm 271(1-2):1-4.
14. Myers R H and Montgomery D C. 1995. Response Surface Methodology: Process and Product Optimization Using Designed Experiments, New York: Wiley-Interscience Publication.
15. Singh Rekhi G, Nellore R V, Hussain A S, Tillman L G. Malinowski, H. J. and Augsburger, L. L. 1999. Identification of critical formulation and processing variables for metoprolol tartrate extended-release (ER) matrix tablets. J Contr Rel 59(3):327-342.
16. US Pharmacopeia XXV. 2001. Drug Release. United States Pharmacopeial Convention, Rockville, Md., 724.
17. Pillay V and Fassihi R. 1998. Evaluation and comparison of dissolution data derived from different modified release dosage forms: an alternative method. J Contr Rel 55:45-55.
18. Taha M O, Aiedah K M, Al-Hiari Y, and Al-Khatib H. 2005. Synthesis of zinc-crosslinked thiolated alginic acid and their in vitro evaluation as potential enteric delivery system with folic acid as model drug. Pharmazie 60(10):736-742.
19. Pillay V and Fassihi R. 1999. In vitro release modulation from crosslinked pellets for site-specific drug delivery to the gastrointestinal tract: I. Comparison of pH-responsive drug release and associated kinetics. J Contr Rel 59 (2):229-242.
20. Swarbrick J (editor). 2004. Encyclopedia of Pharmaceutical Technology, 2^{nd} ed. (update supplement), New York: Marcel Dekker, p. 241-242.
21. Sriamornsak. P. 1999. Effect of calcium concentration, hardening agent and drying condition on release characteristics of oral proteins from calcium pectinate gel beads. Eur J Pharm Sci 8(3):221-227.
22. Guo J H. 1993. Effects of plasticizers on water permeation and mechanical properties of cellulose acetate: antiplasticization in slightly plasticized polymer film. Drug Dev Ind Pharm 19:1541-1555.
23. Saettone M F, Perini G, Rijli P, Rodriguez L and Cini M. 1995. Effect of different polymer-plasticizer combinations on ‘in vitro’ release of theophylline from coated pellets. Int J Pharm 126:83-88.
24. Sastry S V, Wilber W, Reddy I K and Khan M A. 1998. Aqueous-based polymeric dispersion: preparation and characterization of cellulose acetate pseudolatex. Int J Pharm 165:175-189.
25. Sinko C M and Amidon G L. 1989. Plasticizer-induced changes in the mechanical rate of response of film coatings: approach to quantitating plasticizer effectiveness. Int J Pharm 55:247
26. Hickey A J. 1996. Inhalation Aerosols: Physical and Biological Basis for Therapy. New York: Marcel Dekker, p. 241-242.
27. Bodmeier R. 1997. Tabletting of Coated Pellets. Eur J Pharm Biopharm 43: 1-8.
28. Fassihi A R. 1998. Consolidation behavior of polymeric substances in non-disintegrating solid matrices. Int J Pharm 44: 249-256.
29. Schmidt C and Bodmeier R. 2001. A multiparticulate drug-delivery system based on pellets incorporated into congealable polyethylene glycol carrier materials. Int J Pharm 216 (1-2): 9-16.
30. du Toit L C Pillay V and Danckwerts M P. 2006. Application of Synergism and Variation in Ionic Compatibilities within a Hydrophilic Polymeric Sodium Starch Glycolate-κ-Carrageenan Combination: Textural Profiling of the Suspension Behavior. J Bioact Compat Polymers, 21: 107-122.

1-60. (canceled)

61. A pharmaceutical dosage form comprising a heterogeneously configured multiparticulate drug delivery system, said heterogeneously configured multiparticulate system comprising a multiplicity of enterosoluble and/or gastrosoluble multiparticulates loaded with at least one active pharmaceutical composition for the site-specific delivery of said active pharmaceutical composition to a specific region in the gastrointestinal tract via human or animal body.

62. The pharmaceutical dosage form as claimed in claim 61, in which at least a portion of the pharmaceutical dosage form is formed from a polymeric material.

63. The pharmaceutical dosage form as claimed in claim 62, wherein the multiparticulates are formed from a polymeric material.

64. The pharmaceutical dosage form as claimed in claim 62, in which the pharmaceutical dosage form is rendered gastroretentive as a result of a process of decreasing the density of the multiparticulates.

65. The pharmaceutical dosage form as claimed in claim 64, in which the pharmaceutical dosage form is rendered gastroretentive as a result of lyophilization.

66. The pharmaceutical dosage form as claimed in claim 64, in which the polymeric material comprises at least one pH-sensitive polymer demonstrating solubility in intestinal fluid above a pH of 4.0.

67. The pharmaceutical dosage form as claimed in claim 66, in which the pH-sensitive polymer interacts and swells minimally in the presence of water at low pH, and ionises, swells and dissolves in water at high pH.

68. The pharmaceutical dosage form as claimed in claim 67, in which the pH-sensitive polymer is partially neutralized to form a latex, and is salted-out and crosslinked in an electrolyte or salt solution with electrolytes or salts chosen from the Hofmeister Series of salts.

69. The pharmaceutical dosage form as claimed in claim 66, in which the pH-sensitive polymer comprises a polymethacrylate-type polymer that is crosslinked to form a series of heterogeneously configured multiparticulates.

70. The pharmaceutical dosage form as claimed in claim 66, in which the pH-sensitive polymer is carboxylated and contains mixed acid and ester functional groups.

71. The pharmaceutical dosage form as claimed in claim 66, in which the pH-sensitive polymer possesses acidic side groups and demonstrates at least partial solubility in aqueous solutions.

72. The pharmaceutical dosage form as claimed in claim 71, in which the pH soluble polymer is at least partially soluble in at least one aqueous solution selected from the group consisting of water, buffered salt solutions, or alkaline solutions.

73. The pharmaceutical dosage form as claimed in claim 71, in which the acidic side groups comprise a carboxylic acid moeity possessing the propensity to interact with suitable cations.

74. The pharmaceutical dosage form as claimed in claim 66, in which the pH-sensitive polymer comprises at least one enteric polymer possessing carboxylic acid and ester groups on the polymer backbone.

75. The pharmaceutical dosage form as claimed in claim 74, in which the enteric polymer is selected from the group consisting of: methacrylic acid-based polymers, phthalate-based enteric polymers, and hydroxypropyl methylcellulose acetate succinate, and wherein the at least one pH-sensitive polymer further comprises a poly(methacrylic acid-co-ethylacrylate) copolymer.

76. The pharmaceutical dosage form as claimed in claim 75, in which the methacrylic acid-based polymer is selected from methacrylic acid and ethyl acrylate copolymers, and methacrylic acid and methyl methacrylate copolymers.

77. The pharmaceutical dosage form as claimed in claim 75, in which the phthalate-based enteric polymer is selected from cellulose acetate phthalate and polyvinyl acetate phthalate.

78. The pharmaceutical dosage form as claimed in claim 61, in which the active pharmaceutical composition is an acid-sensitive active pharmaceutical composition selected from the group consisting of: an active pharmaceutical composition which is unstable or degraded at acidic pH; an active pharmaceutical composition affecting gastric performance; an active pharmaceutical composition which causes local irritation of the gastric mucosa; an active pharmaceutical composition for which intestinal targeting is required for attainment of adequate concentrations in the lower gastrointestinal tract and bioavailability; and an active pharmaceutical composition which accelerates the degradation of other active pharmaceutical compositions in the gastrointestinal tract.

79. The pharmaceutical dosage form as claimed in claim 78, wherein the acid-sensitive active pharmaceutical composition which is unstable or degraded at acidic pH comprises a component selected from the group consisting of enzymes, proteins, and macrolide antibiotics.

80. The pharmaceutical dosage form as claimed in claim 79, wherein the macrolide antibiotic is erythromycin.

81. The pharmaceutical dosage form as claimed in claim 78, wherein the active pharmaceutical composition which causes local irritation of the gastric mucosa comprises a component selected from the group consisting of valproic acid, diclofenac, and acetylsalicylic acid.

82. The pharmaceutical dosage form as claimed in claim 78, wherein the active pharmaceutical composition for which intestinal targeting is required for attainment of adequate concentrations in the lower gastrointestinal tract and bioavailability comprises a component selected from the group consisting of 5-aminosalicylic acid, prodrugs of mesalazine, and prodrugs of sulfasalazine.

83. The pharmaceutical dosage form as claimed in claim 78, wherein the active pharmaceutical composition which accelerates the degradation of other active pharmaceutical compositions in the gastrointestinal tract comprises a component selected from the group consisting of isoniazid, rifampicin, pyrazinamide, didanosine, and ketoconazole

84. The pharmaceutical dosage form as claimed in claim 61, in which the active pharmaceutical composition is a standard regimental therapy selected from the group consisting of fixed dose combinations, antiretroviral therapy, and tuberculosis regimens.
